(19) United States
(12) Patent Application Publication
(10) Pub. No.: US 2023/0128770 A1
(43) Pub. Date: Apr. 27, 2023

(54) COMPOSITIONS AND METHODS FOR ENHANCING DIFFERENTIATION OF STEM CELL-DERIVED BETA CELLS

(71) Applicant: President and Fellows of Harvard College, Cambridge, MA (US)

(72) Inventors: Douglas A. Melton, Cambridge, MA (US); Edwin Rosado-Olivieri, Somerville, MA (US)

(21) Appl. No.: 17/770,281

(22) PCT Filed: Mar. 31, 2020
(86) PCT No.: PCT/US2020/026041

§ 371 Date: Apr. 19, 2022

Related U.S. Application Data
(60) Provisional application No. 62/924,139, filed on Oct. 21, 2019.

(51) Int. Cl. C12N 5/071; C07K 11/02; A61P 3/10

(52) U.S. Cl. CPC C12N 5/0692; C07K 11/02; A61P 3/10

(57) ABSTRACT

Disclosed herein are methods for generating stem cell-derived beta cells and compositions including stem cell-derived beta cells.

### RELATED APPLICATIONS

This application claims the benefit of U.S. Provisional Application No. 62/924,139, filed on Oct. 21, 2019, the contents of which are hereby incorporated by reference in its entirety.

### BACKGROUND OF THE INVENTION

β cell loss is a hallmark of type I and type II diabetes, and cell replacement strategies have been explored to restore functional β cells [1,2]. Recently, approaches to direct the differentiation of hPSCs into endocrine cells have been demonstrated [3,4], providing an alternate source of β cells for cell replacement therapies, drug discovery, and disease modeling. While these protocols are based on developmental signals involved in in vivo pancreatic development, the understanding of how these signaling factors coordinate the last steps of β-cell differentiation is incomplete [5,6].

### SUMMARY OF THE INVENTION

The Hippo signaling pathway controls the proliferation and specification of pancreatic progenitors into the endocrine lineage. Downregulation of YAP, an effector of the pathway, enhances endocrine progenitor differentiation and the generation of SC-β cells with improved insulin secretion, as well as reducing the presence of proliferative progenitor cells.

Disclosed herein are methods of producing a stem cell-derived beta cell. The methods comprise contacting at least one endocrine progenitor cell during a beta cell differentiation protocol with a YAP modulator, wherein the at least one endocrine progenitor cell differentiates into at least one stem cell-derived beta cell.

In some embodiments, the YAP modulator is a YAP inhibitor and/or a YAP activator. In some embodiments, the YAP modulator is a small molecule or an shRNA. In some embodiments, the YAP modulator is verteporfin or roscovitine.

In some embodiments, the at least one endocrine progenitor cell is contacted with the YAP modulator between or during days 9-25 of a beta cell differentiation protocol. In some embodiments, the at least one endocrine progenitor cell is contacted with the YAP modulator between days 9-14, 14-18, or 18-25 of the beta cell differentiation protocol. In some embodiments, the at least one endocrine progenitor cell is contacted with the YAP modulator during at least one of Stage 4, Stage 5, and the first seven days of Stage 6 of a beta cell differentiation protocol.

In some embodiments, the at least one endocrine progenitor cell is contacted with 0.1 μM to 0.5 μM of the YAP modulator. In some embodiments, the at least one endocrine progenitor cell is contacted with 0.3 μM to 0.4 μM of the YAP modulator.

In some embodiments, the at least one endocrine progenitor cell is derived from an iPS cell, an ES cell, and/or a fibroblast.

In some embodiments, the stem cell-derived beta cell exhibits increased expression (e.g., 1.5-fold to 2-fold increased expression) of at least one of NGN3 (NEUROG3), PAX6, NEUROD1, and insulin, as compared to a control sample (e.g., a sample of stem cell-derived beta cells obtained without using a YAP modulator).

Also disclosed herein is a population of stem cell-derived beta cells produced according to the methods described herein.

In some embodiments, the population comprises a 1.5-fold to 3-fold increase in C-peptide+/NKX6.1+ stem cell-derived beta cells, as compared to a control sample. In some embodiments, the stem cell-derived beta cells in the population exhibit 1.5-fold to 2-fold increased expression of at least one of NGN3 (NEUROG3), PAX6, NEUROD1, and insulin, as compared to a control sample. In some embodiments, the population comprises a 2-fold to 3-fold reduction in proliferative progenitor cells (e.g., SOX9+ ductal like proliferative progenitor cells and/or Ki67+ proliferative cells), as compared to a control sample

Disclosed herein are methods comprising modulating YAP expression during a beta cell differentiation protocol. In some embodiments, a method comprises increasing YAP expression during a beta cell differentiation protocol. In some embodiments, YAP expression is increased during Stage 4 (e.g., days 9 to 14) of a beta cell differentiation protocol. In some embodiments, a method comprises decreasing YAP expression during a beta cell differentiation protocol. In some embodiments, YAP expression is decreased during Stage 5 (e.g., days 14 to 18) and/or the first seven days of Stage 6 (e.g., days 18 to 25) of a beta cell differentiation protocol.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: ncbi.nlm nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-H demonstrates YAP downregulation in SC-endocrine and insulin-producing β cells. FIG. 1A provides a diagram of the directed differentiation of hPSCs into insulin-producing β cells. FIGS. 1B-1E provide immunofluorescence micrographs of YAP expression in PDX1+ early pancreatic progenitors, NKX6.1+ late pancreatic progenitors, NGN3+ endocrine progenitors, and C-peptide+β cells. Representative images and cropped blots (bottom panels) are shown. Scale bar: 50 μm. White arrows denote NKX6.1+/YAP1− (FIG. 1C), NGN3+/YAP1− (FIG. 1D), and C-peptide+/NKX6.1+/YAP1− cells (FIG. 1E). FIG. 1F shows proportion of cells expressing YAP (green), C-peptide (purple) and CHGA (blue) from stage 3 through stage 6 as estimated by flow cytometry. FIGS. 1G-1I show proportion of YAP+ and YAP− cells of all NKX6.1+ (FIG. 1G), CHGA+ (FIG. 1H) and CHGA− (FIG. 1I) cells as quantified by flow cytometry. hPSCs human pluripotent stem cells, DE definitive endoderm, GTE gut tube endoderm, PP1 pancreatic progenitor 1, PP2 pancreatic progenitor 2, EN endocrine precursor, 13 insulin-producing β cells. Data represent mean values±SEM, ****p<0.0001, two-sided student's t test (n=3 biologically independent samples per group)

FIGS. 2A-2N demonstrate YAP activity regulates the specification and proliferation of NKX6.1+ progenitors. FIG. 2A provides a diagram of experimental design for FIGS. 2B-2G. FIGS. 2B-2D provide flow cytometry of PDX1 and NKX6.1 expression in DMSO (FIG. 2B) or veterporfin-treated (FIG. 2C) MPPs and quantification of the proportion of PDX1/NKX6.1 co-positive pancreatic progenitors (FIG. 2D) from FIGS. 2B and 2C, as assayed at the end of stage 4. FIGS. 2E-2G provide flow cytometry analysis of PDX1 and the proliferation marker Ki67 in DMSO (FIG. 2E) and veterporfin-treated pancreatic progenitors (FIG. 2F), and quantification of coexpression of PDX1 and Ki67 in MPPs from FIG. 2E and FIG. 2F (FIG. 2G) as assayed at the end of stage 4. FIG. 2H provides experimental design for FIGS. 2I-2N. FIGS. 2I-2J show effects of YAP shRNA expression during pancreatic differentiation on the expression of YAP and target genes assayed by qPCR for RNA (FIG. 2I) and western blot for protein (FIG. 2J), as assayed at the end of stage 4. qPCR values normalized to the average expression value of shControl samples. FIGS. 2K-2N show effects of the expression of non-targeting control (FIG. 2K) and YAP shRNAs (FIG. 2L) during pancreatic progenitor differentiation on NKX6.1 and CHGA expression and quantification of the proportion of NKX6.1+ (FIG. 2M) and NKX6.1−/CHGA+ (FIG. 2N) cells from FIG. 2I and FIG. 2J, as assayed at the end of stage 4. Data represent mean±SEM, **p<0.01, ***p<0.001, ****p<0.0001, two-sided student's t test (n=3 biologically independent samples per group)

FIGS. 3A-3L demonstrate YAP inhibition enhances the generation of endocrine progenitors and SC-β cells. FIG. 3A provides experimental design for FIGS. 3B-3G. FIGS. 3B-3D provide flow cytometry analysis of NGN3 expression at stage 5, day 3 of DMSO and verteporfin-treated pancreatic progenitors and proportion of NGN3+ cells from FIG. 3B and FIG. 3C. FIGS. 3E-3G provide flow cytometry analysis of NKX6.1 and C-peptide expression upon completion of the differentiation protocol of DMSO and verteporfin-treated pancreatic progenitors and quantification of the proportion of C-peptide+/NKX6.1+ SC-β cells from FIG. 3E and FIG. 3F. FIG. 3H show effects of non-targeting control and YAP shRNAs during endocrine differentiation on lineage marker mRNAs assayed by Nanostring assayed at the end of stage 5. Differentially expressed genes (adjusted p-value <0.05) are displayed with genes relevant to endocrine induction highlighted in red. Data presented as z-scores. FIGS. 3I-3K provide flow cytometry of C-peptide and NKX6.1 expression in non-targeting control and YAP shRNA-expressing cells and proportion of cells co-expressing both markers from FIGS. 3I-3J at the end of stage 6 differentiation (stage 6 day 14). FIG. 3L show proportion of cells expressing YAP in cultures of cells expressing non-targeting control and YAP shRNAs assayed at the end of stage 6 differentiation (stage 6 day 14) by flow cytometry. Data represent mean±SEM, *p<0.05, ***p<0.001, two-sided student's t test (n=3 biologically independent samples per group).

FIGS. 4A-4N demonstrate YAPS6A overexpression impairs differentiation of β cells and promotes proliferation. FIG. 4A provides experimental workflow for the lentiviral overexpression of a stabilized form of YAP (YAPS6A) during β-cell differentiation. FIGS. 4B-4C provide flow cytometry of YAP expression and quantification of YAP+ cells in LacZ control and YAP-overexpressing stem cell-derived cells during β-cell differentiation (assayed at stage 6, day 14). FIGS. 4D-4F provide flow cytometry analysis and quantification of C-peptide and NKX6.1 coexpression in LacZ and YAPS6A-overexpressing cells at the end of stage 6 differentiation (stage 6, day 14). FIGS. 4G-4J provide flow cytometry analysis and quantification of C-peptide and Ki67 expression in LacZ and YAPS6A-overexpressing cells. FIGS. 4K-4N show quantification of EdU staining (FIGS. 4K-4L) and cleaved Caspase-3 staining (FIGS. 4M-4N) in monohormonal C-peptide+ and C-peptide− cells by flow cytometry at the end of stage 6 differentiation (stage 6, day 14). Data represent mean (center line)±min to max (bounds of box), **p<0.01, ***p<0.001, ****p<0.0001, two-sided student's t test (n=3 biologically independent samples per group).

FIGS. 5A-5D demonstrate functionality of SC-β cells upon YAP inhibition and overexpression. FIGS. 5A-5B show insulin secretion levels for DMSO or verteporfin-treated stem cell-derived β cells during sequential rounds of glucose and KCl challenge and insulin secretion stimulation indexes. Insulin secretion levels were normalized to total insulin content for each sample. Stimulation indexes were calculated as a ratio of insulin secretion at high glucose (20 mM) relative to the basal secretion (2.8 mM glucose). FIGS. 5C-5D show insulin secretion levels of LacZ and YAPS6A-overexpressing stem cell-derived β cells during sequential rounds of glucose and KCl stimulation and insulin secretion stimulation indexes. Data represent mean±SEM, **p<0.01, ***p<0.001, ****p<0.0001, n.s. non-significant, two-sided student's t test, VTPF verterporfin (n=3 biologically independent samples per group).

FIGS. 6A-6N demonstrate YAP inhibition reduces Sox9+ progenitors in vitro. FIG. 6A provides experimental design for FIGS. 6B-6E. FIGS. 6B-6D provide flow cytometry of SOX9 expression in the control and verteporfin-treated differentiating progenitors and quantification of the proportion of SOX9+ cells from FIG. 6B and FIG. 6C at the end of stage 6 differentiation (stage 6, day 14). mCFP is an unstained control. Data represent mean±SEM, **p<0.01 (n=3 biologically independent samples per group). FIG. 6E shows proportion of Ki67+ in control or verteporfin-treated differentiating progenitors as assayed by flow cytometry at the end of stage 6 differentiation. Data represent mean±SEM, ***p<0.001 (n=3 biologically independent samples per group). FIGS. 6F-6H provide flow cytometry analysis of SOX9 in non-targeting control and YAP shRNA-expressing pancreatic progenitors and quantification of SOX9+ cell proportion (FIG. 6H) at the end of stage 6 differentiation. Data represent mean±SEM, ***p<0.001 (n=3 biologically independent samples per group). FIG. 6I provides experimental design for FIGS. 6J-6N. FIGS. 6J-6K show in vivo GSIS of control and verteporfin-derived SC-β cell grafts as assayed by serum human insulin before and 30 min after a glucose injection 8 weeks post transplantation. FIGS. 6L-6M provides immunofluorescence micrographs of grafts 12 weeks post transplantation of control and verteporfin-derived SC-β cell grafts stained for C-peptide and SOX9. Representative images and cropped blots (bottom panels) are shown. Asterisk (*) denotes nonspecific staining. Scale bar: 50 μm. FIG. 6N shows quantification of SOX9+ of DAPI+ cells within grafts 12 weeks after transplantation. Data represent mean (center line)±min to max (whiskers) and lower and upper quartiles (bounds of box), ***p<0.001, two-sided student's t test (n=4 animals per group, values correspond to the average proportion of SOX9+ cells from four histological sections per animal).

FIGS. 7A-7I demonstrate YAP expression in progenitor cells during endocrine differentiation. FIG. 7A provides histogram plots of YAP expression of cells collected from stage 3 through stage 6 of 13 cell differentiation, assayed by flow cytometry. Highlighted area in grey denotes gate on YAP-negative cells. FIGS. 7B-7C provide flow cytometry analysis of co-expression of YAP with NKX6.1 (FIG. 7B) and CHGA (FIG. 7C) at multiple stages of 13 cell differentiation. FIG. 7D provides immunohistological analysis of YAP and CHGA expression in pancreatic progenitors (end of stage 4). Arrows indicate a downregulated expression of YAP in CHGA+ endocrine cells. FIG. 7E provides immunofluorescent micrographs of YAP, NGN3, and CHGA in differentiating endocrine progenitors (stage 5, day 3). FIGS. 7F-7G provide immunohistological analysis of nuclear, cytoplasmic or downregulated expression of YAP in NKX6.1+ (FIG. 7F) and NGN3+ cells (FIG. 7G) at stage 4, 5 and 6. FIGS. 7H-7I show coexpression of YAP and SOX9 in progenitors after endocrine induction as assessed by flow cytometry (FIG. 7E) and immunohistochemistry (FIG. 7F). Scale bar: 50 μm. Data represent mean±SEM, ****p<0.0001, two-sided student's t-test (n=3 biologically independent samples per group). St4c: stage 4 complete, St5c: stage 5 complete.

FIGS. 8A-8F demonstrate cell cycle inhibition does not enhance endocrine differentiation. FIG. 8A provides qPCR analysis of NGN3 expression of verteporfin and roscovitine-treated progenitors at stage 5, day 4. qPCR values normalized to the average expression value of control samples. FIG. 8B shows proportion of NGN3+ cells in roscovitine-treated differentiating MPPs as assayed by flow cytometry at stage 5, day 4. FIGS. 8C-8F provide flow cytometry analysis of CHGA and NKX6.1 expression upon completion of the endocrine specification stage (stage 5) and quantification of the proportion of CHGA+/NKX6.1+ cells in control, verteporfin- and roscovitine treated differentiations. Data represent mean±SEM, *p<0.05, ****p<0.0001, two-sided student's t-test (n=3 biologically independent samples per group).

FIGS. 9A-9J demonstrate enhanced endocrine differentiation upon YAP inhibition. FIG. 9A provides gating strategy and staining control for FIGS. 9B-9E and FIGS. 3E-3F. FIGS. 9B-9E show staining control and replicate data for control and verteporfin-treated differentiations collected at the end of stage 6 differentiation (stage 6 day 14). FIGS. 9F-9J provide flow cytometry analysis of C-peptide, Glucagon and Somatostatin expression and quantification of the proportion of monohormonal endocrine cells in control and verteporfin-treated differentiations. Data represent mean±SEM, *p<0.05, ***p<0.001, two-sided student's t-test (n=3 biologically independent samples per group).

FIGS. 10A-10H demonstrate YAP inhibition enhances endocrine differentiation of multiple hPSC cell lines. FIGS. 10A-10D provide flow cytometry and quantification of C-peptide and NKX6.1 expression of control and verteporfin-treated differentiations performed with the 1016 and 13B iPSC cell lines collected at the end of stage 6 (stage 6 day 14). FIGS. 10E-10H provide flow cytometry and quantification of CHGA and NKX6.1 expression at the end of stage 6 (stage 6 day 14). Data presented as a fold increase over stage-matched control differentiations. *p<0.05, **p<0.01, two-sided t-test on log fold changes (n=3 biologically independent samples per group).

FIGS. 11A-11D demonstrate insulin secretion and insulin content measurements of SC-β cells. FIGS. 11A-11B show raw insulin secretion values for stage-matched control, verteporfin-treated and YAPS6A-overexpressing β cells during sequential stimulations with low and high glucose, and KCl depolarization. FIGS. 11C-11D show insulin content measurements normalized to total number of cells of stage matched SC-β cells. Data represent mean±SEM, two-sided student's t-test (n=3 biologically independent samples per group). n.s.: not-significant.

FIGS. 12A-12H demonstrate YAPS6A inhibits SC-β cell differentiation. FIGS. 12A-12C provide flow cytometry analysis and quantification of C-peptide and NKX6.1 expression of LacZ or YAPS6A-overexpressing SC-β cells differentiated in the presence of verteporfin and collected at the end of stage 6 differentiation (stage 6 day 14). FIG. 12D provides experimental design for FIGS. 12E-12H. Expression of YAPS6A and GFP was transiently induced with doxycycline during the first 4 days of stage 6 (Dox ON), followed by 8 days with no doxycycline added (Dox OFF). FIGS. 12E-12F provide flow cytometry analysis and quantification of C-peptide and NKX6.1 expression as well as EdU staining of SC-β cells collected at stage 6 day 12 as outlined in d. EdU pulse was performed for 4 hours and 4 days after YAPS6A/GFP induction. FIGS. 12G-12H show insulin secretion during sequential stimulations with low and high glucose, and KCl depolarization of SC-β cells after a transient overexpression of YAPS6A or GFP as outlined in FIG. 12D. Data represent mean±SEM. *p<0.05, **p<0.01, two-sided student's t-test (n=3 biologically independent samples per group). n.s.: non-significant, DOX: doxycycline.

FIG. 13 provides a list of taqman probes used for qPCR analysis.

### DETAILED DESCRIPTION OF THE INVENTION

Stem cell-derived insulin-producing beta cells (SC-β) offer an inexhaustible supply of functional β cells for cell replacement therapies and disease modeling for diabetes. While successful directed differentiation protocols for this cell type have been described, the mechanisms controlling its differentiation and function are not fully understood. Here it is shown that the Hippo pathway controls the proliferation and specification of pancreatic progenitors into the endocrine lineage. Downregulation of YAP, an effector of the pathway, enhances endocrine progenitor differentiation and the generation of SC-β cells with improved insulin secretion. A chemical inhibitor of YAP acts as an inducer of endocrine differentiation and reduces the presence of proliferative progenitor cells. Conversely, sustained activation of YAP results in impaired differentiation, blunted glucose-stimulated insulin secretion, and increased proliferation of SC-β cells. Together these results support a role for YAP in controlling the self-renewal and differentiation balance of pancreatic progenitors and limiting endocrine differentiation in vitro.

Aspects of the disclosure relate to compositions, methods, kits, and agents for generating stem cell-derived beta cells (referred to herein as non-naturally occurring beta cells, non-native beta cells, or mature beta cells) from at least one stem cell, and beta cells produced by those compositions, methods, kits, and agents for use in cell therapies, assays, and various methods of treatment.

The methods described herein exhibit numerous advantages, including enhanced differentiation of SC-β cells, as well as depletion of progenitor-like cells (e.g., ductal-like and proliferative cells) from the population of differentiated cells. The in vitro-produced beta cells generated according to the methods described herein demonstrate many advantages, including improved insulin secretion. In addition, the generated beta cells may provide a new platform for cell therapy (e.g., transplantation into a subject in need of additional and/or functional beta cells) and research.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro. Unless otherwise indicated the methods described herein can be performed both in vivo and in vitro.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans that secrete two hormones, insulin and glucagon.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “endocrine progenitor,” “pancreatic progenitor,” or “pancreatic precursor” are used interchangeably herein and refer to a stem cell which is capable of forming any of pancreatic endocrine cells, pancreatic exocrine cells, or pancreatic duct cells. The term “pdx1-positive pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “pdx1-positive, NKX6-1-positive pancreatic progenitor” as used herein refers to a cell which is a pancreatic endoderm (PE) cell. A pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “mature stem cell-derived beta cell”, “SC-beta cell”, and “stem cell-derived beta cell” refer to cells (e.g., pancreatic β cells) that display at least one marker indicative of a pancreatic β cell, express insulin, and display a GSIS response characteristic of an endogenous mature β cell. In some embodiments, the “SC-β cell” comprises a mature pancreatic β cells. It is to be understood that the SC-β cell need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner).

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

As used herein, the term “proliferation” means growth and division of cells. In some embodiments, the term “proliferation” as used herein in reference to cells refers to a group of cells that can increase in number over a period of time.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a beta cell precursors), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, shRNAs, lipoproteins, aptamers, and modifications and combinations thereof etc. In certain embodiments, agents are small molecules having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one endocrine progenitor cell with a maturation or differentiation factor, or combination of maturation or differentiation factors) is intended to include incubating the differentiation medium and/or agent and the cell together in vitro (e.g., adding the maturation or differentiation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one endocrine progenitor cell with a differentiation factor as in the embodiments described herein can be conducted in any suitable manner. For example, the cells may be treated in adherent culture, or in suspension culture. The disclosure contemplates any conditions which promote the formation of stem cell-derived beta cells. Examples of conditions that promote the formation of stem cell-derived beta cells include, without limitation, suspension culture in low attachment tissue culture plates, spinner flasks, aggrewell plates. In some embodiments, the inventors have observed that stem cell-derived beta cells have remained stable in media containing 20% serum (e.g., heat inactivated fetal bovine serum).

It is understood that the cells contacted with a maturation or differentiation factor can also be simultaneously or subsequently contacted with another agent, such as a growth factor or other differentiation agent or environments to stabilize the cells, or to differentiate the cells further.

Similarly, at least one endocrine progenitor cell can be contacted with at least one maturation factor and then contacted with at least another maturation factor. In some embodiments, the cell is contacted with at least one maturation factor, and the contact is temporally separated, and in some embodiments, a cell is contacted with at least one maturation factor substantially simultaneously. In some embodiments, the cell is contacted with at least two, at least three, at least four, at least five, at least six, at least seven, at least eight, at least nine, or at least 10 maturation factors.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a mature beta cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of stem cell-derived beta cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not stem cell-derived beta cells as defined by the terms herein. In some embodiments, the present invention encompasses methods to expand a population of stem cell-derived beta cells, wherein the expanded population of stem cell-derived beta cells is a substantially pure population of stem cell-derived beta cells.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

As used herein, the term “xenogeneic” refers to cells that are derived from different species.

As used herein, the term “DNA” is defined as deoxyribonucleic acid.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms “treat”, “treating”, “treatment”, etc. refer to providing medical or surgical attention, care, or management to an individual. The individual is usually ill or injured, or at increased risk of becoming ill relative to an average member of the population and in need of such attention, care, or management. It may include administering to a subject an effective amount of a composition so that the subject exhibits a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. The term “treatment” includes prophylaxis. Those in need of treatment include those already diagnosed with a condition (e.g., muscle disorder or disease), as well as those likely to develop a condition due to genetic susceptibility or other factors.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion. The phrases “systemic administration,” “administered systemically”, “peripheral administration” and “administered peripherally” as used herein mean the administration of stem cell-derived cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease,” “reduced,” “reduction,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing beta cells (e.g., SC-β cells) or precursors thereof, germ cells may be used in place of, or with, the stem cells to provide at least one beta cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006). One possible application of ES cells is to generate new pancreatic beta cells for the cell replacement therapy of type I diabetics, by first producing endoderm, e.g., definitive endoderm, from, e.g., hESCs, and then further differentiating the definitive endoderm into at least one insulin-positive endocrine cell or precursor thereof, then further differentiating or maturing the at least one insulin-positive endocrine cell or precursor thereof into a stem cell-derived beta cell.

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1: Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one maturation factor according to the methods as disclosed herein, whereas in other examples it may be desirable to differentiate the stem cells to one or more intermediate cell types prior to exposure of the at least one maturation factor (s) or agent(s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESB GN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into stem cell-derived beta cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497; Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic microenvironment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Alternatively, pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin Typically, enzymatic digestion is halted before cells become completely dispersed (˜5 min with collagenase IV). Clumps of ˜10 to 2,000 cells are then plated directly onto the substrate without further dispersal.

Generating Stem Cell-Derived Beta Cells

Aspects of the disclosure relate to generating stem cell-derived beta cells (e.g., a population of differentiated cells comprising stem cell-derived beta cells). Generally, stem cell-derived beta cells or precursors thereof, e.g., endocrine progenitor cells produced according to the methods disclosed herein demonstrate several hallmarks of functional beta cells, including, but not limited to, exhibiting a GSIS response.

The stem cell-derived beta cells can be produced according to any suitable culturing protocol or series of culturing protocols to differentiate a stem cell or pluripotent cell to a desired stage of differentiation. In some embodiments, the stem cell-derived beta cells or the precursors thereof are produced by culturing at least one pluripotent cell for a period of time and under conditions suitable for the at least one pluripotent cell to differentiate into the stem cell-derived beta cells or the precursors thereof.

In some embodiments, the stem cell-derived beta cells or precursors thereof are maintained in culture by methods known by one of ordinary skill in the art, and in some embodiments, propagated prior to being converted into stem cell-derived beta cells by the methods as disclosed herein.

Further, stem cell-derived beta cells or precursors thereof, e.g., endocrine progenitor cells can be from any mammalian species, with non-limiting examples including a murine, bovine, simian, porcine, equine, ovine, or human cell. For clarity and simplicity, the description of the methods herein refers to a mammalian stem cell-derived beta cell or precursor thereof, but it should be understood that all of the methods described herein can be readily applied to other cell types of stem cell-derived beta cells or precursors thereof. In some embodiments, the stem cell-derived beta cells or precursors thereof are derived from a human individual.

In some embodiments stem cell-derived beta cells may be produced using any protocol known to those of skill in the art, including the methods disclosed in WO 2015/002724 and WO 2014/201167, as well as in Veres et al. (2019) Nature 569, 368-373, Rezania et al. (2014) Nature Biotechnology 32(11):1121-33, Nair et al. (2019) Nature Cell Biology 21(2):263-274, Russ et al. (2015) EMBO J. 34, 1759-177, and Velazco-Cruz et al. (2019) 12(2), 351-365, all of which are incorporated herein by reference.

In some embodiments, definitive endoderm cells can be obtained by differentiating at least some pluripotent cells in a population into definitive endoderm cells, e.g., by contacting a population of pluripotent cells with at least one growth factor from the TGF-β superfamily (e.g., Activin A), and a WNT signaling pathway activator (e.g., Chir99021), to induce the differentiation of at least some of the pluripotent cells into definitive endoderm cells, wherein the definitive endoderm cells express at least one marker characteristic of definitive endoderm cells. In certain embodiments, at least one growth factor from the TGF-β superfamily (e.g., 100 ng/mL of Activin A) and a WINT signaling pathway activator (e.g., 3 μM Chir99021) are administered on day 1 of a differentiation protocol. In certain embodiments, at least one growth factor from the TGF-β superfamily (e.g., 100 ng/mL Activin A) is administered on day two of the differentiation protocol. In some embodiments, a suitable culture medium for differentiating pluripotent cells into definitive endoderm cells comprises S1 media.

In some embodiments, primitive gut tube cells can be obtained by differentiating at least some definitive endoderm cells in a population into primitive gut tube cells, e.g., by contacting definitive endoderm cells with at least one growth factor from the fibroblast growth factor (FGF) family (e.g., KGF), to induce the differentiation of at least some of the definitive endoderm cells into primitive gut tube cells, wherein the primitive gut tube cells express at least one marker characteristic of primitive gut tube cells. In certain embodiments, at least one growth factor from the fibroblast growth factor (FGF) family (e.g., 50 ng/mL of KGF) is administered on days 4 and 6 of the differentiation protocol. In some embodiments, a suitable culture medium for differentiating definitive endoderm cells into primitive gut tube cells comprises S2 media.

In some aspects, Pdx 1-positive pancreatic progenitor cells can be obtained by differentiating at least some primitive gut tube cells in a population into Pdx1-positive pancreatic progenitor cells, e.g., by contacting primitive gut tube cells with at least one bone morphogenic protein (BMP) signaling pathway inhibitor (e.g., LDN193189), at least one growth factor from the FGF family (e.g., KGF), at least one Sonic hedgehog (SHH) pathway inhibitor (e.g., Sand), at least one retinoic acid (RA) signaling pathway activator (e.g., RA), and at least one protein kinase C activator (e.g., PdBU), to induce the differentiation of at least some of the primitive gut tube cells into Pdx1-positive pancreatic progenitor cells, wherein the Pdx1-positive pancreatic progenitor cells express Pdx1. In certain embodiments, at least one bone morphogenic protein (BMP) signaling pathway inhibitor (e.g., 200 nM LDN193189), at least one growth factor from the FGF family (e.g., 50 ng/mL KGF), at least one SHH pathway inhibitor (e.g., 0.25 μM Sand), at least one retinoic acid (RA) signaling pathway activator (e.g., 2 μM RA), and at least one protein kinase C activator (e.g., 500 nM PdBU) are administered on day 7 of the differentiation protocol. In certain embodiments, at least one growth factor from the FGF family (e.g., 50 ng/mL KGF), at least one SHH pathway inhibitor (e.g., 0.25 μM Sand), at least one RA signaling pathway activator (e.g., 2 μM RA), and at least one protein kinase C activator (e.g., 500 nM PdBU) are administered on day 8 of the differentiation protocol. In some embodiments, a suitable culture medium for differentiating primitive gut tube cells into Pdx1-positive pancreatic progenitor cells comprises S3 media.

In some aspects, a method of producing a NKX6-1-positive pancreatic progenitor cell from a Pdx 1-positive pancreatic progenitor cell comprises contacting a population of cells comprising Pdx1-positive pancreatic progenitor cells with at least one growth factor from the fibroblast growth factor (FGF) family (e.g., KGF), a sonic hedgehog (SHH) pathway inhibitor (e.g., Sand), a retinoic acid (RA) signaling pathway activator (e.g., RA), a Rock inhibitor (e.g., Y27632), and at least one growth factor from the TGF-β superfamily (e.g., Activin A), to induce the differentiation of at least one Pdx1-positive pancreatic progenitor cell in the population into NKX6-1-positive pancreatic progenitor cells, wherein the NX6-1-positive pancreatic progenitor cells expresses NKX6-1. In certain embodiments, at least one growth factor from the fibroblast growth factor (FGF) family (e.g., 50 ng/mL KGF), a sonic hedgehog (SHH) pathway inhibitor (e.g., 0.25 μM Sand), a retinoic acid (RA) signaling pathway activator (e.g., 100 nM RA), a Rock inhibitor (e.g., 10 μM Y27632), and at least one growth factor from the TGF-β superfamily (e.g., 5 ng/mL Activin A) are administered on days 9, 11, and 13 of the differentiation protocol. In some embodiments, a suitable culture medium for differentiating Pdx1-positive pancreatic progenitor cells into NKX6-1 positive pancreatic progenitor cells comprises S3 media.

In some aspects, a method of producing an insulin-positive endocrine cell from an NKX6-1-positive pancreatic progenitor cell comprises contacting a population of cells comprising NKX6-1-positive pancreatic progenitor cells with a TGF-β signaling pathway inhibitor (e.g., Alk5 inhibitor II), a thyroid hormone signaling pathway activator (e.g., T3), a SHH pathway inhibitor (e.g., Sand), a RA signaling pathway activator (e.g., RA), a γ-secretase inhibitor (e.g., XXI), and at least one growth factor from the epidermal growth factor (EGF) family (e.g., Betacellullin), to induce the differentiation of at least one NX6-1-positive pancreatic progenitor cell in the population into an insulin-positive endocrine cell, wherein the insulin-positive endocrine ceil expresses insulin. In certain embodiments, a TGF-β signaling pathway inhibitor (e.g., 10 μM Alk5 inhibitor II), a thyroid hormone signaling pathway activator (e.g., 1 μM T3), a SHH pathway inhibitor (e.g., 0.25 μM Sand), a RA signaling pathway activator (e.g., 100 nM RA), a γ-secretase inhibitor (e.g., 1 μM XXI), and at least one growth factor from the epidermal growth factor (EGF) family (e.g., 20 ng/mL Betacellullin) are administered on days 14 and 16 of the differentiation protocol. In certain embodiments, a TGF-β signaling pathway inhibitor (e.g., 10 μM Alk5 inhibitor II), a thyroid hormone signaling pathway activator (e.g., 1 μM T3), a RA signaling pathway activator (e.g., 25 nM RA), a γ-secretase inhibitor (e.g., 1 μM XXI), and at least one growth factor from the epidermal growth factor (EGF) family (e.g., 20 ng/mL Betacellullin) are administered on days 18 and 20 of the differentiation protocol. In some embodiments, a suitable culture medium for differentiating NKX6-1-positive pancreatic progenitor cells into insulin-positive endocrine cell comprises S5 media.

In some aspects, a method of producing a stem cell-derived beta cell from an insulin-positive endocrine cell comprises culturing a population of insulin-positive endocrine cells in a suitable culture medium (e.g., S3). In certain embodiments, a population of insulin-positive endocrine cells is cultured for 21-35 days in S3 media.

In some embodiments, the disclosure provides methods for generating stem cell-derived beta cells from endocrine progenitor cells, the method comprising differentiating at least one endocrine progenitor cell into a stem cell-derived beta cell, wherein, during the differentiation process, the Hippo signaling pathway is manipulated (e.g., inhibited) to enhance endocrine progenitor differentiation and the generation of stem cell-derived beta cells having improved insulin secretion. In some embodiments, the at least one endocrine progenitor cell is contacted with a YAP modulator (e.g., a YAP inhibitor) during the differentiation process. In some embodiments, a population of cells comprising endocrine progenitor cells is contacted with at least one YAP modulator (e.g., YAP inhibitor).

The disclosure contemplates the use of any YAP inhibitor that enhances the differentiation of endocrine progenitor cells and results in the generation of stem cell-derived beta cells that display improved insulin secretion (e.g., alone or in combination with another YAP modulator). In some embodiments, the YAP modulator is a YAP inhibitor. In some embodiments, the YAP modulator is a YAP activator. In some embodiments, YAP is overexpressed during the differentiation of the endocrine progenitor cells. In some embodiments, a YAP modulator comprises a small molecule, nucleic acid, amino acid, metabolite, polypeptide, antibody and antibody-like molecules, aptamers, macrocycles, or other molecules. In some embodiments a population of endocrine progenitor cells can be genetically altered by transfection or transduction with a suitable vector, homologous recombination, or other appropriate technique, so that they express a gene of interest. In some embodiments, a YAP modulator comprises a shRNA. In some embodiments, a YAP modulator comprises verteporfin. In some embodiments, a YAP modulator comprises roscovitine. In some embodiments, YAP is overexpressed via transient induction of YAPS61 (e.g., via lentiviral transfection). In some embodiments, a YAP modulator is one such as those described in Johnson et al. (2013) Nature Reviews Drug Discovery 13, 63-79 (e.g., modulators listed in Table 2); Davis et al. (2011) Nature Biotechnology 29, 1046-1051 (e.g., CEP-701/lestaurtanib as an activator of YAP via LATS1/2 inhibition); and Fan et al. (2016) Science Translational Medicine Vol. 8, Issue 352, pp. 352ra108 (e.g., XMU-MP-1 as an activator of YAP via MST1/2 inhibition), incorporated herein by reference.

In some embodiments, a YAP inhibitor is administered for a period of time during Stages 4-6 of a beta cell differentiation protocol, e.g., for a period of time during days 9-25 of a beta cell differentiation protocol disclosed herein. In some aspects, a YAP inhibitor is administered during Stage 4 of a beta cell differentiation protocol, e.g., during days 9-14 of a beta cell differentiation protocol disclosed herein. In some aspects, a YAP inhibitor is administered during Stage 5 of a beta cell differentiation protocol, e.g., during days 14-18 of a beta cell differentiation protocol disclosed herein. In some aspects, a YAP inhibitor is administered during Stage 6 of a beta cell differentiation protocol, e.g., during days 18-25 of a beta cell differentiation protocol disclosed herein. In some embodiments, a YAP inhibitor is administered during the entirety of Stage 5 of a beta cell differentiation protocol. In some embodiments, a YAP inhibitor is administered is during the first seven days of Stage 6 of a beta cell differentiation protocol. In some embodiments, a YAP inhibitor is administered during the entirety of Stage 5 and during the first seven days of Stage 6 of a beta cell differentiation protocol.

In some embodiments, 0.1 μM to 15 μM YAP inhibitor is administered during a beta cell differentiation process. In some embodiments, 0.5 μM to 10 μM, 1.0 to 7.5 μM, or 3 μM to 5 μM YAP inhibitor is administered during a beta cell differentiation process. In some embodiments, 0.1 μM to 0.5 μM or 0.3 μM to 0.4 μM YAP inhibitor is administered during a beta cell differentiation process. In some embodiments, 0.35 μM YAP inhibitor is administered during a beta cell differentiation process. In some embodiments, 5 μM to 15 μM or 7.5 μM to 13 μM YAP inhibitor is administered during a beta cell differentiation process. In some embodiments, 10 μM YAP inhibitor is administered during a beta cell differentiation process.

In some embodiments, a YAP modulator (e.g., a YAP activator) is administered during Stage 4 of a beta cell differentiation protocol, e.g., during days 9-14 of a beta cell differentiation protocol disclosed herein. In some aspects, YAP is overexpressed during Stage 4 of a beta cell differentiation protocol, e.g., during days 9-14 of a beta cell differentiation protocol disclosed herein. In some embodiments, YAP is overexpressed via lentiviral transduction. In some embodiments, expression of YAP is induced during Stage of a beta cell differentiation protocol to induce NKX6.1 expression and promote proliferation/expansion of the NKX6.1 progenitors.

In some embodiments, YAP expression is increased during Stage 4 of a beta cell differentiation protocol and YAP expression is decreased during Stages 5 and 6 of a beta cell differentiation protocol.

In some embodiments, contacting endocrine progenitor cells with a YAP inhibitor during differentiation, e.g., in accordance with a method described herein, enhances the differentiation of the endocrine progenitor cells into stem cell-derived beta cells. In some embodiments, a YAP inhibitor enhances the differentiation of endocrine progenitor cells into stem cell-derived beta cells, wherein the stem cell-derived beta cells exhibit increased expression of NGN3 (NEUROG3), PAX6, NEUROD1, and insulin, e.g., in accordance with a method described herein. In some embodiments, expression of NGN3 (NEUROG3), PAX6, NEUROD1, and/or insulin is increased at least 1-fold, 1.25-fold, 1.5-fold, 1.75-fold, 2-fold, 2.25-fold, or 2.5-fold over a control or reference sample. In some embodiments, expression of NGN3 (NEUROG3), PAX6, NEUROD1, and/or insulin is increased 1.25-fold to 2.25-fold, or 1.5-fold to 2-fold as compared to a control or reference sample. A control sample may be a population of cells prepared using a beta cell differentiation protocol without a YAP modulator (e.g., absent a YAP inhibitor or YAP activator).

In some embodiments, a YAP inhibitor reduces the presence of proliferative progenitor cells (e.g., SOX9+ ductal like progenitor cells and/or Ki67+ proliferating cells) in a population of stem cell-derived beta cells, e.g., obtained in accordance with a method described herein. In some embodiments, the presence of SOX9+ ductal like proliferative cells is decreased in a population of stem cell-derived beta cells by at least 1.5-fold, 1.75-fold, 2-fold, 2.25-fold, 2.5-fold, 2.75-fold, t 3-fold, 3.25-fold, or 3.5-fold as compared to a control or reference sample. In some embodiments, the presence of SOX9+ ductal like proliferative cells is decreased in a population of stem cell-derived beta cells by 1.5-fold to 3.5-fold or by 2-fold to 3-fold as compared to a control or reference sample. In some embodiments, the presence of Ki67 proliferative cells is decreased in a population of stem cell-derived beta cells by at least 1.5-fold, 1.75-fold, 2-fold, 2.25-fold, 2.5-fold, 2.75-fold, 3-fold, 3.25-fold, or 3.5-fold as compared to a control or reference sample. In some embodiments, the presence of Ki67 proliferative cells is decreased in a population of stem cell-derived beta cells by 1.5-fold to 3.5-fold or by 2-fold to 3-fold as compared to a control or reference sample. In some embodiments, the presence of SOX9+ ductal like proliferative cells and Ki67 proliferative cells is decreased in a population of stem cell-derived beta cells by 2-fold to 3-fold as compared to a control or reference sample.

In some embodiments, a YAP inhibitor enhances the differentiation of endocrine progenitor cells into C-peptide+/NKX6.1+ stem cell-derived beta cells, e.g., obtained in accordance with a method described herein. In some embodiments, the use of a YAP inhibitor during a beta cell differentiation protocol results in at least a 1.25-fold, 1.5-fold, 1.75-fold, 2-fold, 2.25-fold, 2.5-fold, 2.75-fold, 3-fold, 3.25-fold, or 3.5-fold increase in C-peptide+/NKX6.1+ stem cell-derived beta cells in a population of stem cell-derived beta cells, as compared to a control or reference sample. In some embodiments, the use of a YAP inhibitor during a beta cell differentiation protocol results in a 1-fold to 3.5-fold or a 1.5-fold to 3-fold increase in C-peptide+/NKX6.1+ stem cell-derived beta cells in a population of stem cell-derived beta cells, as compared to a control or reference sample.

In some embodiments, a YAP activator or overexpression of YAP promotes expression of NKX6.1 in a population of endocrine progenitor cells, e.g., in a population of pancreatic progenitor cells. In some embodiments, the use of a YAP activator during a beta cell differentiation protocol results in at least a 1.25-fold, 1.5-fold, 1.75-fold, 2-fold, 2.25-fold, 2.5-fold, 2.75-fold, 3-fold, 3.25-fold, or 3.5-fold increase in NKX6.1+ progenitor cells in a population of endocrine progenitor cells, as compared to a control or reference sample. In some embodiments, the use of a YAP activator during a beta cell differentiation protocol results in a 1-fold to 3.5-fold or a 1.5-fold to 2.5-fold increase in NKX6.1+ progenitor cells in a population of endocrine progenitor cells, as compared to a control or reference sample.

Aspects of the disclosure involve generating stem cell-derived beta cells which resemble endogenous pancreatic beta cells in form and function, but nevertheless are distinct from native pancreatic beta cells.

Generating stem cell-derived beta cells by differentiating at least one endocrine progenitor cell using the methods of the disclosure has a number of advantages. First, the methods of the disclosure allow one to generate autologous stem cell-derived beta cells, which are cell specific to and genetically matched with an individual. In general, autologous cells are less likely than non-autologous cells to be subject to immunological rejection. The cells are derived from at least one endocrine progenitor cell, e.g., an insulin-positive endocrine cell obtained by reprogramming a somatic cell (e.g., a fibroblast) from the individual to an induced pluripotent state, and then culturing the pluripotent cells to differentiate at least some of the pluripotent cells to at least one endocrine progenitor cell, followed by transplantation of the at least one endocrine progenitor cell into the individual such that the at least one endocrine progenitor cell thereof matures in vivo into a stem cell-derived beta cell, or induced maturation in vitro of the at least one endocrine progenitor cell into a stem cell-derived beta cell.

In some embodiments, a subject from which at least one endocrine progenitor cell is obtained is a mammalian subject, such as a human subject. In some embodiments, the subject is suffering from diabetes. In such embodiments, the at least one endocrine progenitor cell can be differentiated into a stem cell-derived beta cell ex vivo by the methods as described herein and then administered to the subject from which the cells were harvested in a method to treat the subject for diabetes.

In some embodiments, the stem cell-derived beta cells are a substantially pure population of stem cell-derived beta cells. In some embodiments, a population of stem cell-derived beta cells or precursors thereof comprises a mixture of pluripotent cells or differentiated cells. In some embodiments, a population of stem cell-derived beta cells or precursors thereof comprises a mixture of proliferative progenitor cells. In some embodiments, a population of stem cell-derived beta cells or precursors thereof is substantially free or devoid of embryonic stem cells or pluripotent cells or iPS cells.

Stem Cell-Derived Beta Cells

In some embodiments, the disclosure provides stem cell-derived beta cells. The stem cell-derived beta cells disclosed herein share many distinguishing features of native beta cells, but are different in certain aspects (e.g., gene expression profiles). In some embodiments, the stem cell-derived beta cell is non-native or non-naturally occurring. As used herein, “non-native” or “non-naturally occurring” means that the beta cell (e.g., stem cell-derived beta cell) is markedly different in certain aspects from beta cells which exist in nature, i.e., native beta cells. In should be appreciated, however, that these marked differences typically pertain to structural features which may result in the stem cell-derived beta cells exhibiting certain functional differences, e.g., although the gene expression patterns of stem cell-derived beta cells differ from native beta cells, the stem cell-derived beta cells behave in a similar manner to native beta cells but certain functions may be altered (e.g., improved) compared to native beta cells.

The stem cell-derived beta cells of the disclosure share many characteristic features of native beta cells which are important for normal beta cell function. In some embodiments, the stem cell-derived beta cell exhibits a glucose stimulated insulin secretion (GSIS) response in vitro. In some embodiments, the stem cell-derived beta cell exhibits a GSIS response in vivo. In some embodiments, the stem cell-derived beta cell exhibits in vitro and in vivo GSIS responses. In some embodiments, the GSIS responses resemble the GSIS responses of an endogenous pancreatic β cell. In some embodiments, the stem cell-derived beta cell exhibits a GSIS response to at least one glucose challenge. In some embodiments, the stem cell-derived beta cell exhibits a GSIS response to at least two sequential glucose challenges. In some embodiments, the stem cell-derived beta cell exhibits a GSIS response to at least three sequential glucose challenges. In some embodiments, the GSIS responses resemble the GSIS response of endogenous human islets to multiple glucose challenges. In some embodiments, the GSIS response is observed immediately upon transplanting the cell into a human or animal. In some embodiments, the GSIS response is observed within approximately 24 hours of transplanting the cell into a human or animal. In some embodiments, the GSIS response is observed within approximately one week of transplanting the cell into a human or animal. In some embodiments, the GSIS response is observed within approximately two weeks of transplanting the cell into a human or animal. In some embodiments, the stimulation index of the cell as characterized by the ratio of insulin secreted in response to high glucose concentrations compared to low glucose concentrations is similar to the stimulation index of an endogenous mature pancreatic β cell. In some embodiments, the stem cell-derived beta cell exhibits a stimulation index of greater than 1. In some embodiments, the stem cell-derived beta cell exhibits a stimulation index of greater than or equal to 1. In some embodiments, the stem cell-derived beta cell exhibits a stimulation index of greater than 1.1. In some embodiments, the stem cell-derived beta cell exhibits a stimulation index of greater than or equal to 1.1. In some embodiments, the stem cell-derived beta cell exhibits a stimulation index of greater than 2. In some embodiments, the stem cell-derived beta cell exhibits a stimulation index of greater than or equal to 2. In some embodiments, the stem cell-derived beta cell exhibits a stimulation index of at least 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or greater.

In some embodiments, the stem cell-derived beta cell exhibits cytokine-induced apoptosis in response to cytokines. In some embodiments, the stem cell-derived beta cell exhibits cytokine-induced apoptosis in response to a cytokine selected from the group consisting of interleukin-1β (IL-β), interferon-γ (INF-γ), tumor necrosis factor-α (TNF-α), and combinations thereof.

In some embodiments, insulin secretion from the stem cell-derived beta cell is enhanced in response to known anti-diabetic drugs (e.g., anti-diabetic drugs which act on β cells ex vivo or in vitro, and/or anti-diabetic drugs generally in vivo). The disclosure contemplates any known anti-diabetic drug. In some embodiments, insulin secretion from the stem cell-derived beta cell is enhanced in response to a secretagogue. In some embodiments, the secretagogue is selected from the group consisting of an incretin mimetic, a sulfonylurea, a meglitinide, and combinations thereof.

In some embodiments, the stem cell-derived beta cell is monohormonal. In some embodiments, the stem cell-derived beta cell exhibits a morphology that resembles the morphology of an endogenous pancreatic β cell. In some embodiments, the stem cell-derived beta cell encapsulates crystalline insulin granules. In some embodiments, the stem cell-derived beta cell exhibits encapsulated crystalline insulin granules under electron microscopy that resemble insulin granules of an endogenous pancreatic β cell. In some embodiments, the stem cell-derived beta cell exhibits a low rate of replication. In some embodiments, the stem cell-derived beta cell exhibits a low rate of replication. In some embodiments, the stem cell-derived beta cell exhibits a low, but increased rate of replication as measured by staining for C-peptide and Ki67 in response to treatment with prolactin.

In some embodiments, the stem cell-derived beta cell increases intracellular Ca′ in response to glucose. In some embodiments, the stem cell-derived beta cell exhibits a glucose stimulated Ca²⁺ flux (GSCF) that resembles the GSCF of an endogenous pancreatic β cell. In some embodiments, the stem cell-derived beta cell exhibits a GSCF response to at least three sequential glucose challenges in a manner that resembles the GSCF response of an endogenous pancreatic β cell to multiple glucose challenges.

In some embodiments, the stem cell-derived beta cell expresses at least one marker characteristic of an endogenous pancreatic β cell selected from the group consisting of insulin, C-peptide, PDX1, MAFA, NKX6-1, PAX6, NEUROD1, glucokinase (GCK), SLC2A1, PCSK1, KCNJ11, ABCC8, SLC30A8, SNAP25, RAB3A, GAD2, and PTPRN. In certain embodiments, the stem cell-derived beta cell exhibits increased expression of at least one of NGN3 (NEUROG3), PAX6, NEUROD1, and Muslin.

In some embodiments, the stem cell-derived beta cell does not express at least one marker (e.g., a marker not expressed by endogenous pancreatic β cells) selected from the group consisting of a) a hormone selected from the group consisting of i) glucagon (GCG), and ii) somatostatin (SST); b) an acinar cell marker selected from the group consisting of i) amylase, and ii) carboxypeptdase A (CPA1), c) an a cell marker selected from the group consisting of i) GCG, Arx, Irx1, and Irx2, d) a ductal cell marker selected from the group consisting of i) CFTR, and ii) Sox9.

The stem cell-derived beta cells are differentiated in vitro from any starting cell as the invention is not intended to be limited by the starting cell from which the stem cell-derived beta cells are derived. Exemplary starting cells include, without limitation, endocrine progenitor cells, such as an insulin-positive endocrine cell, multipotent pancreatic progenitor cells (MPPs) (e.g., a Nkx6-1-positive pancreatic progenitor cell and/or a Pdx1-positive pancreatic progenitor cell), a pluripotent stem cell, an embryonic stem cell, and an induced pluripotent stem cell. In some embodiments, the stem cell-derived beta cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the stem cell-derived beta cells disclosed herein can be differentiated in vitro from an endocrine progenitor cell. In some embodiments, the stem cell-derived beta cell is differentiated in vitro from a precursor selected from the group consisting of an insulin-positive endocrine cell, a Nkx6-1-positive pancreatic progenitor cell, a Pdx1-positive pancreatic progenitor cell, and a pluripotent stem cell. In some embodiments, the pluripotent stem cell is selected from the group consisting of an embryonic stem cell and induced pluripotent stem cell. In some embodiments, the stem cell-derived beta cell or the pluripotent stem cell from which the stem cell-derived beta cell is derived is human. In some embodiments, the stem cell-derived beta cell is human.

In some embodiments, the stem cell-derived beta cell is not genetically modified. In some embodiments, the stem cell-derived beta cell obtains the features it shares in common with native β cells in the absence of a genetic modification of cells. In some embodiments, the stem cell-derived beta cell is genetically modified.

In some embodiments, the insulin produced per stem cell-derived beta cell is at least 0.5 μIU per 1000 cells per 30 minute incubation (e.g., ex vivo) at a high glucose concentration.

In some embodiments, the insulin produced per stem cell-derived beta cell is at least 1, at least 2, at least 3, at least 4 at least 5 at least 6, at least 7 at least 8 or at least 9 μIU per 1000 cells per 30 minute incubation at a high glucose concentration. In some embodiments, the insulin produced per stem cell-derived beta cell is between 0.5 and 10 μIU per 1000 cells per 30 minute incubation at a high glucose concentration. In some embodiments, the insulin produced per stem cell-derived beta cell is approximately 2.5 μIU per 1000 cells per 30 minute incubation at a high glucose concentration.

In some aspects, the disclosure provides a cell line comprising a stem cell-derived beta cell described herein. In some embodiments, the stem cell-derived beta cells stably express insulin. In some embodiments, the stem cell-derived beta cell can be frozen, thawed, and amplified with a doubling time of 24 to 44 hours without significant morphological changes until at least 30 passages.

Aspects of the disclosure relate to isolated populations of stem cell-derived beta cells produced according to methods described herein. In some embodiments, a population of stem cell-derived beta cells is produced by contacting at least one endocrine progenitor cell with at least one, at least two, at least three, at least four, at least five, at least six, at least seven, at least eight, at least nine, or at least ten differentiation factors described herein. In some embodiments, a population of stem cell-derived beta cells is produced by contacting at least one endocrine progenitor cell with at least one differentiation factor (e.g., at least one, at least two, at least three, at least four, at least five, at least six, at least seven, at least eight, at least nine, or at least ten differentiation factors) and at least one YAP modulator described herein (e.g., a YAP inhibitor and/or a YAP activator).

Aspects of the disclosure involve microcapsules comprising isolated populations of cells described herein (e.g., stem cell-derived beta cells). Microcapsules are well known in the art. Suitable examples of microcapsules are described in the literature (e.g., Orive et al., “Application of cell encapsulation for controlled delivery of biological therapeutics”, Advanced Drug Delivery Reviews (2013), dx.doi.org/10.1016/j.addr.2013.07.009; Hernandez et al., “Microcapsules and microcarriers for in situ cell delivery”, Advanced Drug Delivery Reviews 2010; 62:711-730; Murua et al., “Cell microencapsulation technology: Towards clinical application”, Journal of Controlled Release 2008; 132:76-83; and Zanin et al., “The development of encapsulated cell technologies as therapies for neurological and sensory diseases”, Journal of Controlled Release 2012; 160:3-13). Microcapsules can be formulated in a variety of ways. Exemplary microcapsules comprise an alginate core surrounded by a polycation layer covered by an outer alginate membrane. The polycation membrane forms a semipermeable membrane, which imparts stability and biocompatibility. Examples of polycations include, without limitation, poly-L-lysine, poly-L-ornithine, chitosan, lactose modified chitosan, and photopolymerized biomaterials. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the stem cell-derived beta cell is modified with PEG before being encapsulated within alginate. In some embodiments, the isolated populations of cells, e.g., stem cell-derived beta cells are encapsulated in photoreactive liposomes and alginate. It should be appreciated that the alginate employed in the microcapsules can be replaced with other suitable biomaterials, including, without limitation, PEG, chitosan, PES hollow fibers, collagen, hyaluronic acid, dextran with RGD, EHD and PEGDA, PMBV and PVA, PGSAS, agarose, agarose with gelatin, PLGA, and multilayer embodiments of these.

In some embodiments, compositions comprising populations of stem cell-derived beta cells produced according to the methods described herein can also be used as the functional component in a mechanical device. For example, a device may contain a population of stem cell-derived beta cells (e.g., produced from populations of endocrine progenitor cells) behind a semipermeable membrane that prevents passage of the cell population, retaining them in the device. Other examples of devices include those contemplated for either implantation into a diabetic patient, or for extracorporeal therapy.

Aspects of the disclosure involve assays comprising isolated populations of stem cell-derived beta cells described herein. In some embodiments, the assays can be used for identifying one or more candidate agents which promote or inhibit a stem cell-derived beta cell fate. In some embodiments, the assays can be used for identifying one or more candidate agents which promote or enhance the differentiation of at least one endocrine progenitor cell into stem cell-derived beta cells.

The disclosure contemplates methods in which stem cell-derived beta cells are generated according to the methods described herein from iPS cells derived from cells extracted or isolated from individuals suffering from a disease (e.g., diabetes, obesity, or a β cell-related disorder), and those stem cell-derived beta cells are compared to normal β cells from healthy individuals not having the disease to identify differences between the stem cell-derived beta cells and normal β cells which could be useful as markers for disease (e.g., epigenetic and/or genetic). In some embodiments, β cells are obtained from a diabetic individual and compared to normal β cells, and then the β cells are reprogrammed to iPS cells and the iPS cells are analyzed for genetic and/or epigenetic markers which are present in the β cells obtained from the diabetic individual but not present in the normal β cells, to identify markers (e.g., pre-diabetic). In some embodiments, the iPS cells and/or stem cell-derived cells derived from diabetic patients are used to screen for agents (e.g., agents which are able to modulate genes contributing to a diabetic phenotype).

Confirmation of the Presence and the Identification of Stem Cell-Derived Beta Cells

One can use any means common to one of ordinary skill in the art to confirm the presence of a stem cell-derived beta cell produced by the differentiation of at least one endocrine progenitor cell by exposure to at least one differentiation factor and at least one YAP modulator as described herein.

In some embodiments, the presence of beta cell markers, e.g. chemically induced beta cells, can be done by detecting the presence or absence of one or more markers indicative of an endogenous beta cell. In some embodiments, the method can include detecting the positive expression (e.g. the presence) of a marker for mature beta cells. In some embodiments, the marker can be detected using a reagent, e.g., a reagent for the detection of NKX6-1 and C-peptide. In particular, mature stem cell-derived beta cells herein express NKX6-1 and C-peptide, and do not express significant levels of other markers which would be indicative of immature beta cells (e.g., MafB).

A reagent for a marker can be, for example, an antibody against the marker or primers for a RT-PCR or PCR reaction, e.g., a semi-quantitative or quantitative RT-PCR or PCR reaction. Such markers can be used to evaluate whether a stem cell-derived beta cell has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one endocrine progenitor cell to a stem cell-derived beta cell (e.g., a mature stem cell-derived beta cell) can be monitored by determining the expression of markers characteristic of mature beta cells. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of stem cell-derived beta cells as well as the lack of significant expression of markers characteristic of endocrine progenitor cells from which it was derived is determined.

As described in connection with monitoring the production of a stem cell-derived beta cell from an endocrine progenitor cell, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as quantitative-PCR by methods ordinarily known in the art. Additionally, techniques for measuring extracellular marker content, such as ELISA, may be utilized.

Stem cell-derived beta cells can also be characterized by the down-regulation of markers characteristic of the pluripotent stem from which the stem cell-derived beta cell is induced from. For example, stem cell-derived beta cells derived from pluripotent stem cells may be characterized by a statistically significant down-regulation of the pluripotent stem cell markers alkaline phosphatase (AP), NANOG, OCT-4, SOX-2, SSEA4, TRA-1-60 or TRA-1-81 in the beta cell relative to the expression in the pluripotent stem cell from which it was derived. Other markers expressed by pluripotent cell markers, include but are not limited to alkaline phosphatase (AP); ABCG2; stage specific embryonic antigen-1 (SSEA-1); SSEA-3; SSEA-4; TRA-1-60; TRA-1-81; Tra-2-49/6E; ERas/ECATS, E-cadherin; βIII tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPAS/ECAT2; ECAT3; ECAT6; ECAT7; ECAT8; ECAT9; ECAT10; ECAT15-1; ECAT15-2; Fth117; Sa114; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tc11); DPPA3/Stella; DPPA4; Dnmt3L; Sox15; Stat3; Grb2; SV40 Large T Antigen; HPV16 E6; HPV16 E7, β-catenin, and Bmi1 and other general markers for pluripotency, etc, and at least one or more of these are down regulated by a statistically significant amount in a beta cell as compared to the pluripotent stem cell from which they were derived.

It is understood that the present invention is not limited to those markers listed as beta cell markers herein, and the present invention also encompasses markers such as cell surface markers, antigens, and other gene products including ESTs, RNA (including microRNAs and antisense RNA), DNA (including genes and cDNAs), and portions thereof.

Enrichment, Isolation and Purification of Stem Cell-Derived Beta Cells

Another aspect of the present invention relates to the isolation of a population of stem cell-derived beta cells from a heterogeneous population of cells, such as a mixed population of cells comprising stem cell-derived beta cells and endocrine progenitor cells from which the stem cell-derived beta cells was derived. A population of stem cell-derived beta cells produced by any of the above-described processes can be enriched, isolated and/or purified by using any cell surface marker present on the stem cell-derived beta cells which is not present on the endocrine progenitor cells from which it was derived. Such cell surface markers are also referred to as an affinity tag which is specific for a stem cell-derived beta cell. Examples of affinity tags specific for stem cell-derived beta cells are antibodies, ligands or other binding agents that are specific to a marker molecule, such as a polypeptide, that is present on the cell surface of a stem cell-derived beta cell but which is not substantially present on other cell types (e.g. endocrine progenitor cells). In some processes, an antibody which binds to a cell surface antigen on a stem cell-derived beta cell is used as an affinity tag for the enrichment, isolation or purification of chemically induced (e.g. by contacting with at least one differentiation factor as described herein) stem cell-derived beta cell produced by the methods described herein. Such antibodies are known and commercially available.

The skilled artisan will readily appreciate the processes for using antibodies for the enrichment, isolation and/or purification of stem cell-derived beta cells. For example, in some embodiments, the reagent, such as an antibody, is incubated with a cell population comprising stem cell-derived beta cells, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FITC-conjugated antibody that is capable of binding to the primary antibody. The stem cell-derived beta cells are then washed, centrifuged and resuspended in buffer. The stem cell-derived beta cell suspension is then analyzed and sorted using a fluorescence activated cell sorter (FACS). Antibody-bound, fluorescent reprogrammed cells are collected separately from non-bound, non-fluorescent cells, thereby resulting in the isolation of stem cell-derived beta cells from other cells present in the cell suspension, e.g. endocrine progenitor cells.

In another embodiment of the processes described herein, the isolated cell composition comprising stem cell-derived beta cells can be further purified by using an alternate affinity-based method or by additional rounds of sorting using the same or different markers that are specific for stem cell-derived beta cells. For example, in some embodiments, FACS sorting is used to first isolate a stem cell-derived beta cell which expresses NKX6-1, either alone or with the expression of C-peptide, or alternatively with a β cell marker disclosed herein from cells that do not express one of those markers (e.g. negative cells) in the cell population. A second FAC sorting, e.g. sorting the positive cells again using FACS to isolate cells that are positive for a different marker than the first sort enriches the cell population for reprogrammed cells.

In an alternative embodiment, FACS sorting is used to separate cells by negatively sorting for a marker that is present on most endocrine progenitor cells, but is not present on stem cell-derived beta cells.

In some embodiments of the processes described herein, stem cell-derived beta cells are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above.

In addition to the procedures just described, chemically induced stem cell-derived beta cells may also be isolated by other techniques for cell isolation. Additionally, stem cell-derived beta cells may also be enriched or isolated by methods of serial subculture in growth conditions which promote the selective survival or selective expansion of the stem cell-derived beta cells. Such methods are known by persons of ordinary skill in the art, and may include the use of agents such as, for example, insulin, members of the TGF-beta family, including Activin A, TGF-beta1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin-like growth factors (IGF-I, II) growth differentiation factor (GDF-5, -6, -7, -8, -10, -11, -15), vascular endothelial cell-derived growth factor (VEGF), Hepatocyte growth factor (HGF), pleiotrophin, endothelin, Epidermal growth factor (EGF), beta-cellulin, among others. Other pharmaceutical compounds can include, for example, nicotinamide, glucagon like peptide-I (GLP-1) and II, GLP-1 and 2 mimetibody, Exendin-4, retinoic acid, parathyroid hormone.

Using the methods described herein, enriched, isolated and/or purified populations of stem cell-derived beta cells can be produced in vitro from endocrine progenitor cells (which were differentiated from pluripotent stem cells by the methods described herein). In some embodiments, preferred enrichment, isolation and/or purification methods relate to the in vitro production of human stem cell-derived beta cells from human endocrine progenitor cells, which were differentiated from human pluripotent stem cells, or from human induced pluripotent stem (iPS) cells. In such an embodiment, where stem cell-derived beta cells are differentiated or matured from endocrine progenitor cells, which were previously derived from iPS cells, the stem cell-derived beta cells can be autologous to the subject from whom the cells were obtained to generate the iPS cells.

Using the methods described herein, isolated cell populations of stem cell-derived beta cells are enriched in stem cell-derived beta cell content by at least about 1- to about 1000-fold as compared to a population of cells before the chemical induction of the endocrine progenitor cells. In some embodiments the population of stem cell-derived beta cells is induced, enhanced, enriched, or increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more as compared to a population of cells before the chemical induction of endocrine progenitor cells.

Compositions Comprising Stem Cell-Derived Beta Cells

Some embodiments of the present invention relate to cell compositions, such as cell cultures or cell populations, comprising stem cell-derived beta cells, wherein the stem cell-derived beta cells have been derived from at least one endocrine progenitor cell. In some embodiments, the cell compositions comprise endocrine progenitor cells. In some embodiments, the cell compositions comprise endocrine cells (e.g., insulin-positive endocrine cells). In some embodiments, the cell compositions comprise NKX6-1-pancreatic progenitor cells. In some embodiments, the cell compositions comprise Pdx1-pancreatic progenitor cells. In some embodiments, the cell compositions comprise primitive gut tube cells. In some embodiments, the cell compositions comprise definitive endoderm cells.

In accordance with certain embodiments, the chemically induced stem cell-derived beta cells are mammalian cells, and in a preferred embodiment, such stem cell-derived beta cells are human stem cell-derived beta cells. In some embodiments, the endocrine progenitor cells have been derived from pluripotent stem cells (e.g., human pluripotent stem cells).

Other embodiments of the present invention relate to compositions, such as an isolated cell population or cell culture, comprising stem cell-derived beta cells produced by the methods as disclosed herein. In such embodiments, the stem cell-derived beta cells comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the stem cell-derived beta cell population. In some embodiments, the composition comprises a population of stem cell-derived beta cells which make up more than about 90% of the total cells in the cell population, for example about at least 95%, or at least 96%, or at least 97%, or at least 98% or at least about 99%, or about at least 100% of the total cells in the cell population are stem cell-derived beta cells.

Certain other embodiments of the present invention relate to compositions, such as an isolated cell population or cell cultures, comprising a combination of stem cell-derived beta cells and endocrine progenitor cells from which the stem cell-derived beta cells were derived. In some embodiments, the endocrine progenitor cells from which the stem cell-derived beta cells are derived comprise less than about 25%, less than about 20%, less than about 15%, less than about 10%, less than about 5%, less than about 4%, less than about 3%, less than about 2% or less than about 1% of the total cells in the isolated cell population or culture.

Additional embodiments of the present invention relate to compositions, such as isolated cell populations or cell cultures, produced by the processes described herein and which comprise chemically induced stem cell-derived beta cells as the majority cell type. In some embodiments, the methods and processes described herein produce an isolated cell culture and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% stem cell-derived beta cells.

In another embodiment, isolated cell populations or compositions of cells (or cell cultures) comprise human stem cell-derived beta cells. In other embodiments, the methods and processes as described herein can produce isolated cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% stem cell-derived beta cells. In preferred embodiments, isolated cell populations can comprise human stem cell-derived beta cells. In some embodiments, the percentage of stem cell-derived beta cells in the cell cultures or populations is calculated without regard to the feeder cells remaining in the culture.

Still other embodiments of the present invention relate to compositions, such as isolated cell populations or cell cultures, comprising mixtures of stem cell-derived beta cells and endocrine progenitor cells from which they were differentiated or matured from. For example, cell cultures or cell populations comprising at least about 5 stem cell-derived beta cells for about every 95 endocrine progenitor cells can be produced. In other embodiments, cell cultures or cell populations comprising at least about 95 stem cell-derived beta cells for about every 5 endocrine progenitor cells can be produced. Additionally, cell cultures or cell populations comprising other ratios of stem cell-derived beta cells to endocrine progenitor cells are contemplated. For example, compositions comprising at least about 1 stem cell-derived beta cell for about every 1,000,000, or at least 100,000 cells, or at least 10,000 cells, or at least 1000 cells or 500, or at least 250 or at least 100 or at least 10 endocrine progenitor cells can be produced.

Further embodiments of the present invention relate to compositions, such as cell cultures or cell populations, comprising human cells, including human stem cell-derived beta cells, which displays at least one characteristic of an endogenous mature beta cell.

In preferred embodiments of the present invention, cell cultures and/or cell populations of stem cell-derived beta cells comprise human stem cell-derived beta cells that are non-recombinant cells. In such embodiments, the cell cultures and/or cell populations are devoid of or substantially free of recombinant human mature stem cell-derived beta cells.

YAP Modulators

Aspects of the disclosure involve contacting endocrine progenitor cells with one or more yes-associated protein (YAP) modulators, for example, to enhance the differentiation of endocrine progenitor cells into stem cell-derived beta cells, to reduce the presence of proliferative progenitor cells, or to promote expression of NKX6.1 in a population of endocrine progenitor cells. The term “YAP modulator” refers to an agent that modulates YAP, an effector of the Hippo signaling pathway. A YAP modulator may be an activator of YAP or an inhibitor of YAP. In some embodiments, YAP expression is increased or decreased.

In some embodiments, the YAP modulator is a YAP inhibitor. A YAP inhibitor may be administered for a period of time during Stages 4-6 of a beta cell differentiation protocol, e.g., for a period of time during days 9-25 of a beta cell differentiation protocol disclosed herein. In some aspects, a YAP inhibitor is administered during Stage 4 of a beta cell differentiation protocol, e.g., during days 9-14 of a beta cell differentiation protocol disclosed herein. In some aspects, a YAP inhibitor is administered during Stage 5 of a beta cell differentiation protocol, e.g., during days 14-18 of a beta cell differentiation protocol disclosed herein. In some aspects, a YAP inhibitor is administered during Stage 6 of a beta cell differentiation protocol, e.g., during days 18-25 of a beta cell differentiation protocol disclosed herein.

In some embodiments, the YAP modulator is a YAP activator. A YAP activator may be administered at Stage 4 of a beta cell differentiation protocol, e.g., during days 9-14 of a beta cell differentiation protocol disclosed herein. In some aspects, YAP is overexpressed at Stage 4 of a beta cell differentiation protocol, e.g., during days 9-14 of a beta cell differentiation protocol disclosed herein.

In some embodiments, a YAP inhibitor enhances the differentiation of endocrine progenitor cells into stem cell-derived beta cells, e.g., in accordance with a method described herein. In some embodiments, a YAP inhibitor enhances the differentiation of endocrine progenitor cells into stem cell-derived beta cells having increased expression of NGN3 (NEUROG3), PAX6, NEUROD1, and insulin, e.g., in accordance with a method described herein. In some embodiments, a YAP inhibitor reduces the presence of proliferative progenitor cells (e.g., SOX9+ ductal like progenitor cells and/or Ki67+ proliferating cells) in a population of stem cell-derived beta cells, e.g., obtained in accordance with a method described herein. In some embodiments, a YAP inhibitor enhances the differentiation of endocrine progenitor cells into C-peptide+/NKX6.1+ stem cell-derived beta cells, e.g., obtained in accordance with a method described herein. In some embodiments, a YAP activator or overexpression of YAP promotes expression of NKX6.1 in a population of endocrine progenitor cells, e.g., in a population of pancreatic progenitor cells.

Generally, at least one YAP modulator described herein can be used alone, or in combination with other YAP modulators and differentiation factors, to generate stem cell-derived beta cells according to the methods as disclosed herein. In some embodiments, at least two, at least three, at least four, at least five, at least six, at least seven, at least eight, at least nine, or at least ten YAP modulators described herein are used in the methods of generating stem cell-derived beta cells.

In some embodiments, a YAP modulator comprises a small molecule, nucleic acid, amino acid, metabolite, polypeptide, antibody and antibody-like molecules, aptamers, macrocycles, or other molecules. In some embodiments a population of endocrine progenitor cells can be genetically altered by transfection or transduction with a suitable vector, homologous recombination, or other appropriate technique, so that they express a gene of interest. In some embodiments, a YAP modulator comprises a shRNA. In some embodiments, a YAP modulator comprises verteporfin. In some embodiments, a YAP modulator comprises roscovitine. In some embodiments, YAP is overexpressed via transient induction of YAPS61 (e.g., via lentiviral transfection).

### Compositions and Kits

Described herein are compositions which comprise a cell described herein (e.g., a stem cell-derived beta cell). In some embodiments, the composition also includes a differentiation factor, a YAP modulator, and/or cell culture media. Described herein are also compositions comprising the compounds described herein (e.g., cell culture media comprising one or more of the compounds described herein).

Also described herein are kits for practicing methods disclosed herein and for making stem cell-derived beta cells disclosed herein. In one aspect, a kit includes at least one endocrine progenitor cell, at least one differentiation factor as described herein, and at least one YAP modulator as described herein, and optionally, the kit can further comprise instructions for converting at least one endocrine progenitor cell to a population of stem cell-derived beta cells using a method described herein. In some embodiments, the kit comprises at least two differentiation factors. In some embodiments, the kit comprises at least three differentiation factors. In some embodiments, the kit comprises at least four differentiation factors. In some embodiments, the kit comprises at least two YAP modulators.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce endocrine progenitor cells into stem cell-derived beta cells. Differentiation factors and YAP modulators can be provided in any form, e.g., liquid, dried or lyophilized form. It is preferred that a compound(s) (e.g., differentiation factor and/or YAP modulator) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a differentiation factor and/or YAP modulator) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit can include one or more containers for the composition containing at least one differentiation factor and at least one YAP modulator as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for stem cell-derived beta cells, e.g., for a marker described herein, e.g., a reagent for the detection of stem cell-derived beta cells. Or in some embodiments, the kit can also comprise reagents for the detection of negative markers of stem cell-derived beta cells for the purposes of negative selection of stem cell-derived beta cells or for identification of cells which do not express these negative markers (e.g., stem cell-derived beta cells). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit can include stem cell-derived beta cells derived from the same type of endocrine progenitor cells, for example for the use as a positive cell type control.

Methods of Administering a Cell

In one embodiment, the cells described herein, e.g. a population of stem cell-derived beta cells is transplantable, e.g., a population of stem cell-derived beta cells can be administered to a subject. In some embodiments, the subject who is administered a population of stem cell-derived beta cells is the same subject from whom a pluripotent stem cell used to differentiate into a stem cell-derived beta cell was obtained (e.g. for autologous cell therapy). In some embodiments, the subject is a different subject. In some embodiments, a subject suffering from diabetes such as type I diabetes, or is a normal subject. For example, the cells for transplantation (e.g. a composition comprising a population of stem cell-derived beta cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of stem cell-derived beta cells can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Pharmaceutical Compositions

For administration to a subject, a cell population produced by the methods as disclosed herein, e.g. a population of stem cell-derived beta cells (produced by contacting at least one endocrine progenitor cell with at least one differentiation factor (e.g., any one, two, three, or more differentiation factors as described herein) and at least one YAP modulator (e.g., any one, two, three, or more YAP modulators as described herein) can be administered to a subject, for example in pharmaceutically acceptable compositions. These pharmaceutically acceptable compositions comprise a therapeutically-effective amount of a population of stem cell-derived beta cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., stem cell-derived beta cells, or composition comprising stem cell-derived beta cells of the present invention which is effective for producing some desired therapeutic effect in at least a sub-population of cells in an animal at a reasonable benefit/risk ratio applicable to any medical treatment. For example, an amount of a population of stem cell-derived beta cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

By “treatment”, “prevention” or “amelioration” of a disease or disorder is meant delaying or preventing the onset of such a disease or disorder, reversing, alleviating, ameliorating, inhibiting, slowing down or stopping the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that the desired effect is produced. Routes of administration suitable for the methods of the invention include both local and systemic administration. Generally, local administration results in more of the administered stem cell-derived beta cells being delivered to a specific location as compared to the entire body of the subject, whereas, systemic administration results in delivery of the stem cell-derived beta cells to essentially the entire body of the subject.

In the context of administering a compound treated cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantation between members of different species).

Stem cell-derived beta cells or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

As used herein, the phrase “subject in need of stem cell-derived beta cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of stem cell-derived beta cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional stem cell-derived beta cells. A subject in need of a population of stem cell-derived beta cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of stem cell-derived beta cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

In type 1 diabetes, beta cells are undesirably destroyed by continued autoimmune response. Thus, this autoimmune response can be attenuated by use of compounds that inhibit or block such an autoimmune response. In some embodiments, a composition comprising a population of stem cell-derived beta cells for administration to a subject can further comprise a pharmaceutically active agent which is an immune response modulator. As used herein, the term “immune response modulator” refers to a compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

A composition comprising stem cell-derived beta cells can be administered to the subject at the same time, or at different times as the administration of a pharmaceutically active agent or composition comprising the same. When administrated at different times, the compositions comprising a population of stem cell-derived beta cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of stem cell-derived beta cells and a composition comprising a pharmaceutically active agent are administered in different pharmaceutical compositions, routes of administration can be different. In some embodiments, a subject is administered a composition comprising stem cell-derived beta cells. In other embodiments, a subject is administered a composition comprising a pharmaceutically active agent. In another embodiment, a subject is administered a composition comprising a population of stem cell-derived beta cells mixed with a pharmaceutically active agent. In another embodiment, a subject is administered a composition comprising a population of stem cell-derived beta cells and a composition comprising a pharmaceutically active agent, where administration is substantially at the same time, or subsequent to each other.

Toxicity and therapeutic efficacy of administration of a compositions comprising a population of stem cell-derived beta cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of stem cell-derived beta cells that exhibit large therapeutic indices, are preferred.

The amount of a composition comprising a population of stem cell-derived beta cells can be tested using several well-established animal models. Examples of such models are described in WO 2015/002724 and WO 2014/201167, which are incorporated herein by reference.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. The therapeutically effective dose of a composition comprising a population of stem cell-derived beta cells can also be estimated initially from cell culture assays. A dose may be formulated in animal models in vivo to achieve a secretion of insulin at a concentration which is appropriate in response to circulating glucose in the plasma. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alterations to the treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules include administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of stem cell-derived beta cells as disclosed herein. In one embodiment of the invention, an isolated population of stem cell-derived beta cells as disclosed herein may be used for the production of a pharmaceutical composition, for the use in transplantation into subjects in need of treatment, e.g. a subject that has, or is at risk of developing diabetes, for example, but not limited to, subjects with congenital and acquired diabetes. In one embodiment, an isolated population of stem cell-derived beta cells may be genetically modified. In another aspect, the subject may have or be at risk of diabetes and/or metabolic disorder. In some embodiments, an isolated population of stem cell-derived beta cells as disclosed herein may be autologous and/or allogeneic. In some embodiments, the subject is a mammal, and in other embodiments the mammal is a human.

The use of an isolated population of stem cell-derived beta cells as disclosed herein provides advantages over existing methods because the population of stem cell-derived beta cells can be differentiated from endocrine progenitor cells derived from stem cells, e.g. iPS cells obtained or harvested from the subject administered an isolated population of stem cell-derived beta cells. This is highly advantageous as it provides a renewable source of stem cell-derived beta cells which can be differentiated from stem cells to endocrine progenitor cells by methods commonly known by one of ordinary skill in the art, and then further differentiated by the methods described herein to pancreatic beta-like cells or cells with pancreatic beta cell characteristics, for transplantation into a subject, in particular a substantially pure population of mature pancreatic beta-like cells that do not have the risks and limitations of cells derived from other systems.

In another embodiment, an isolated population of stem cell-derived beta cells (e.g., mature pancreatic beta cells or beta-like cells) can be used as models for studying properties for the differentiation into insulin-producing cells, e.g. to pancreatic beta cells or pancreatic beta-like cells, or pathways of development of cells of endoderm origin into pancreatic beta cells.

In some embodiments, the endocrine progenitor cells or stem cell-derived beta cells may be genetically engineered to comprise markers operatively linked to promoters that are expressed when a marker is expressed or secreted, for example, a marker can be operatively linked to an insulin promoter, so that the marker is expressed when the endocrine progenitor cells mature or differentiate into stem cell-derived beta cells which express and secrete insulin. In some embodiments, a population of stem cell-derived beta cells can be used as a model for studying the differentiation pathway of cells which differentiate into islet beta cells or pancreatic beta-like cells.

In other embodiments, the insulin-producing, glucose responsive cells can be used as models for studying the role of islet beta cells in the pancreas and in the development of diabetes and metabolic disorders. In some embodiments, the stem cell-derived beta cells can be from a normal subject, or from a subject which carries a mutation and/or polymorphism (e.g. in the gene Pdx1 which leads to early-onset insulin-dependent diabetes mellitus (NIDDM)), as well as maturity onset diabetes of the young type 4 (MODY4), which can be used to identify small molecules and other therapeutic agents that can be used to treat subjects with diabetes with a mutation or polymorphism in Pdx1. In some embodiments, the stem cell-derived beta cells may be genetically engineered to correct the polymorphism in the Pdx1 gene prior to being administered to a subject in the therapeutic treatment of a subject with diabetes. In some embodiments, the stem cell-derived beta cells may be genetically engineered to carry a mutation and/or polymorphism.

One embodiment of the invention relates to a method of treating diabetes or a metabolic disorder in a subject comprising administering an effective amount of a composition comprising a population of stem cell-derived beta cells as disclosed herein to a subject with diabetes and/or a metabolic disorder. In a further embodiment, the invention provides a method for treating diabetes, comprising administering a composition comprising a population of stem cell-derived beta cells as disclosed herein to a subject that has, or has increased risk of developing diabetes in an effective amount sufficient to produce insulin in response to increased blood glucose levels.

In one embodiment of the above methods, the subject is a human and a population of stem cell-derived beta cells as disclosed herein is human cells. In some embodiments, the invention contemplates that a population of stem cell-derived beta cells as disclosed herein is administered directly to the pancreas of a subject, or is administered systemically. In some embodiments, a population of stem cell-derived beta cells as disclosed herein can be administered to any suitable location in the subject, for example in a capsule in the blood vessel or the liver or any suitable site where administered the population of stem cell-derived beta cells can secrete insulin in response to increased glucose levels in the subject.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of stem cell-derived beta cells can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher the blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of stem cell-derived beta cells as disclosed herein to a subject in need thereof is associated with improved exercise tolerance or other quality of life measures, and decreased mortality. The effects of cellular therapy with a population of stem cell-derived beta cells can be evident over the course of days to weeks after the procedure. However, beneficial effects may be observed as early as several hours after the procedure, and may persist for several years. In some embodiments, the effects of cellular therapy with a population of stem cell-derived beta cells occur within two weeks after the procedure.

In some embodiments, a population of stem cell-derived beta cells as disclosed herein may be used for tissue reconstitution or regeneration in a human patient or other subject in need of such treatment. In some embodiments compositions of populations of stem cell-derived beta cells can be administered in a manner that permits them to graft or migrate to the intended tissue site and reconstitute or regenerate the functionally deficient area. Special devices are available that are adapted for administering cells capable of reconstituting a population of beta cells in the pancreas or at an alternative desired location. Accordingly, the stem cell-derived beta cells may be administered to a recipient subject's pancreas by injection, or administered by intramuscular injection.

In some embodiments, compositions comprising a population of stem cell-derived beta cells as disclosed herein have a variety of uses in clinical therapy, research, development, and commercial purposes. For therapeutic purposes, for example, a population of stem cell-derived beta cells as disclosed herein may be administered to enhance insulin production in response to increase in blood glucose level for any perceived need, such as an inborn error in metabolic function, the effect of a disease condition (e.g. diabetes), or the result of significant trauma (i.e. damage to the pancreas or loss or damage to islet β cells). In some embodiments, a population of stem cell-derived beta cells as disclosed herein are administered to the subject not only help restore function to damaged or otherwise unhealthy tissues, but also facilitate remodeling of the damaged tissues.

To determine the suitability of cell compositions for therapeutic administration, the population of stem cell-derived beta cells can first be tested in a suitable animal model. At one level, cells are assessed for their ability to survive and maintain their phenotype in vivo. Cell compositions comprising stem cell-derived beta cells can be administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or β-galactosidase); that have been prelabeled (for example, with BrdU or [3H] thymidine), or by subsequent detection of a constitutive cell marker (for example, using human-specific antibody). The presence and phenotype of the administered population of stem cell-derived beta cells can be assessed by immunohistochemistry or ELISA using human-specific antibody, or by RT-PCR analysis using primers and hybridization conditions that cause amplification to be specific for human polynucleotides, according to published sequence data.

A number of animal models for testing diabetes are available for such testing, and are commonly known in the art, for example as disclosed in U.S. Pat. No. 6,187,991 which is incorporated herein by reference, as well as rodent models; NOD (non-obese mouse), BB_DB mice, KDP rat and TCR mice, and other animal models of diabetes as described in Rees et al, Diabet Med. 2005 April; 22(4):359-70; Srinivasan K, et al., Indian J Med. Res. 2007 March; 125(3):451-7; Chatzigeorgiou A, et al., In Vivo. 2009 March-April; 23(2):245-58, which are incorporated herein by reference.

In some embodiments, a population of stem cell-derived beta cells as disclosed herein may be administered in any physiologically acceptable excipient, where the stem cell-derived beta cells may find an appropriate site for replication, proliferation, and/or engraftment. In some embodiments, a population of stem cell-derived beta cells as disclosed herein can be introduced by injection, catheter, or the like. In some embodiments, a population of stem cell-derived beta cells as disclosed herein can be frozen at liquid nitrogen temperatures and stored for long periods of time, being capable of use on thawing. If frozen, a population of stem cell-derived beta cells will usually be stored in a 10% DMSO, 50% FCS, 40% RPMI 1640 medium. Once thawed, the cells may be expanded by use of growth factors and/or feeder cells associated with culturing stem cell-derived beta cells as disclosed herein.

In some embodiments, a population of stem cell-derived beta cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of stem cell-derived beta cells as disclosed herein will be adapted in accordance with the route and device used for administration. In some embodiments, a composition comprising a population of stem cell-derived beta cells can also comprise or be accompanied with one or more other ingredients that facilitate the engraftment or functional mobilization of the stem cell-derived beta cells. Suitable ingredients include matrix proteins that support or promote adhesion of the stem cell-derived beta cells, or complementary cell types, especially endothelial cells. In another embodiment, the composition may comprise resorbable or biodegradable matrix scaffolds.

In some embodiments, a population of stem cell-derived beta cells as disclosed herein may be genetically altered in order to introduce genes useful in insulin-producing cells such as pancreatic β cells, e.g. repair of a genetic defect in an individual, selectable marker, etc., or genes useful in selection against non-insulin-producing cells differentiated from at least one endocrine progenitor cell or for the selective suicide of implanted stem cell-derived beta cells. In some embodiments, a population of stem cell-derived beta cells can also be genetically modified to enhance survival, control proliferation, and the like. In some embodiments a population of stem cell-derived beta cells as disclosed herein can be genetically altered by transfection or transduction with a suitable vector, homologous recombination, or other appropriate technique, so that they express a gene of interest. In one embodiment, a population of stem cell-derived beta cells is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of mature stem cell-derived beta cells. In some embodiments, a population of stem cell-derived beta cells may be genetically altered using vector containing supernatants over a 8-16 h period, and then exchanged into growth medium for 1-2 days. Genetically altered mature stem cell-derived beta cells can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, a population of stem cell-derived beta cells as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, such as somatostatin, glucagon, and other factors.

Many vectors useful for transferring exogenous genes into target stem cell-derived beta cells as disclosed herein are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. In some embodiments, combinations of retroviruses and an appropriate packaging cell line may also find use, where the capsid proteins will be functional for infecting the stem cell-derived beta cells as disclosed herein. Usually, stem cell-derived beta cells and virus will be incubated for at least about 24 hours in the culture medium. In some embodiments, the stem cell-derived beta cells are then allowed to grow in the culture medium for short intervals in some applications, e.g. 24-73 hours, or for at least two weeks, and may be allowed to grow for five weeks or more, before analysis.

Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bc1-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In one aspect of the present invention, a population of stem cell-derived beta cells as disclosed herein is suitable for administering systemically or to a target anatomical site. A population of stem cell-derived beta cells can be grafted into or nearby a subject's pancreas, for example, or may be administered systemically, such as, but not limited to, intra-arterial or intravenous administration. In alternative embodiments, a population of stem cell-derived beta cells of the present invention can be administered in various ways as would be appropriate to implant in the pancreatic or secretory system, including but not limited to parenteral, including intravenous and intraarterial administration, intrathecal administration, intraventricular administration, intraparenchymal, intracranial, intracisternal, intrastriatal, and intranigral administration. Optionally, a population of stem cell-derived beta cells is administered in conjunction with an immunosuppressive agent.

In some embodiments, a population of stem cell-derived beta cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of stem cell-derived beta cells can be administered to a subject the following locations: clinic, clinical office, emergency department, hospital ward, intensive care unit, operating room, catheterization suites, and radiologic suites.

In other embodiments, a population of stem cell-derived beta cells is stored for later implantation/infusion. A population of stem cell-derived beta cells may be divided into more than one aliquot or unit such that part of a population of stem cell-derived beta cells is retained for later application while part is applied immediately to the subject. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties. At the end of processing, the concentrated cells may be loaded into a delivery device, such as a syringe, for placement into the recipient by any means known to one of ordinary skill in the art.

In some embodiments a population of stem cell-derived beta cells can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. In some embodiments, a population of stem cell-derived beta cells may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of stem cell-derived beta cells could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

Pharmaceutical compositions comprising effective amounts of a population of stem cell-derived beta cells are also contemplated by the present invention. These compositions comprise an effective number of stem cell-derived beta cells, optionally, in combination with a pharmaceutically acceptable carrier, additive or excipient. In certain aspects of the present invention, a population of stem cell-derived beta cells is administered to the subject in need of a transplant in sterile saline. In other aspects of the present invention, a population of stem cell-derived beta cells is administered in Hanks Balanced Salt Solution (HBSS) or Isolyte S, pH 7.4. Other approaches may also be used, including the use of serum free cellular media. In one embodiment, a population of stem cell-derived beta cells is administered in plasma or fetal bovine serum, and DMSO. Systemic administration of a population of stem cell-derived beta cells to the subject may be preferred in certain indications, whereas direct administration at the site of or in proximity to the diseased and/or damaged tissue may be preferred in other indications.

In some embodiments, a population of stem cell-derived beta cells can optionally be packaged in a suitable container with written instructions for a desired purpose, such as the reconstitution or thawing (if frozen) of a population of stem cell-derived beta cells prior to administration to a subject.

In one embodiment, an isolated population of stem cell-derived beta cells as disclosed herein is administered with a differentiation agent and a YAP modulator. In one embodiment, the stem cell-derived beta cells are combined with the differentiation agent and YAP modulator to administer into the subject. In another embodiment, the cells are administered separately to the subject from the differentiation agent and YAP modulator. Optionally, if the cells are administered separately from the differentiation agent and YAP modulator, there is a temporal separation in the administration of the cells and the differentiation agent and YAP modulator. The temporal separation may range from about less than a minute in time, to hours or days in time. The determination of the optimal timing and order of administration is readily and routinely determined by one of ordinary skill in the art.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### EXAMPLES

### Example 1—YAP Inhibition Enhances the Differentiation of Functional Stem Cell-Derived Insulin-Producing β Cells

β cell loss is a hallmark of type I and type II diabetes, and cell replacement strategies have been explored to restore functional β cells [1,2]. Recently, approaches to direct the differentiation of hPSCs into endocrine cells have been demonstrated [3,4], providing an alternate source of β cells for cell replacement therapies, drug discovery, and disease modeling. While these protocols are based on developmental signals involved in in vivo pancreatic development, the understanding of how these signaling factors coordinate the last steps of β-cell differentiation is incomplete [5,6].

During pancreatic development, endocrine cells differentiate from multipotent pancreatic progenitors (MPPs) that express NGN3, a factor essential for endocrine specification [7-10]. Similar to what occurs during in vivo organogenesis, treatment with EGFs and thyroid hormone T3, along with BMP, TGF-β, and Notch inhibition, helps drive stem cell-derived pancreatic progenitors into NGN3-expressing endocrine progenitors [3,4]. Cell cycle arrest of these progenitors accompanies their further differentiation to β cells [8,11,12,13].

The in vitro-differentiated β cells express NKX6.1, PDX1, and insulin, among other genes, all of which are key to their glucose-stimulated insulin secretion (GSIS) function, an essential part of controlling glucose homeostasis in vivo [3,4,14,15]. Genetic studies have indicated a prominent role for NKX6.1 in the development of 13 cells from endocrine progenitors [14], and methods to enhance the numbers of pancreatic progenitors that express NKX6.1 from hPSCs have been described [3,4,16,17,18,19]. It is the subsequent step of differentiation, wherein pancreatic progenitors form monohormal β cells, that the signals controlling the differentiation are less well understood. The present study shows that YAP, a member of the Hippo signaling pathway, is involved in controlling the generation of functional β cells from MPPs.

The Hippo pathway has been shown to integrate tissue architecture by balancing progenitor cell self-renewal and differentiation [20]. Inhibition of Hippo signaling results in the nuclear translocation of the downstream effectors YAP and TAZ, which, upon binding to TEAD coactivators, regulate expression of genes involved in progenitor cell proliferation [20,21]. In contrast, sustained activation of the pathway by growth-restrictive signals promotes terminal differentiation of mature cell types by inducing the phosphorylation, cytoplasmic retention, and degradation of YAP/TAZ21. Constitutive activation of YAP/TAZ in the mouse pancreas results in reduced organ size, acute pancreatitis, and impaired endocrine differentiation [22,23]. YAP plays a role in the control of progenitor expansion and maintenance of human fetal and stem cell-derived MPPs by regulating enhancer elements of transcription factors involved in these processes [24]. A recent study showed that mechanotransduction controls YAP activity in MPPs to direct cell fate via integrin signaling [25]. Moreover, the downregulation of YAP has been documented in NGN3+ endocrine progenitors and islet cells [22-25]. However, the extensive loss of tissue architecture as a result of genetic perturbations of the pathway in vivo confounded an analysis of whether or how YAP controls differentiation in pancreatic endocrine lineages.

Taking advantage of the in vitro differentiation of SC-β cells, a role for YAP as a regulator of progenitor self-renewal and differentiation was ascribed. Studies show that YAP regulates the self-renewal of early progenitors and formation of NKX6.1+ pancreatic progenitors. It is further shown that both the chemical and genetic downregulation of YAP enhance endocrine differentiation and the terminal differentiation of functional monohormonal β cells. Finally, the utility of a YAP inhibitor for the depletion of progenitor cells in vitro is demonstrated.

### Results

### YAP is Downregulated During Endocrine Differentiation

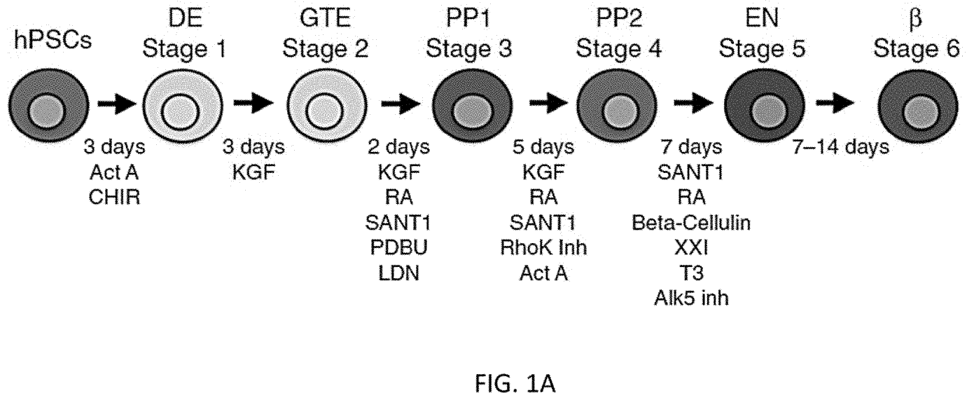

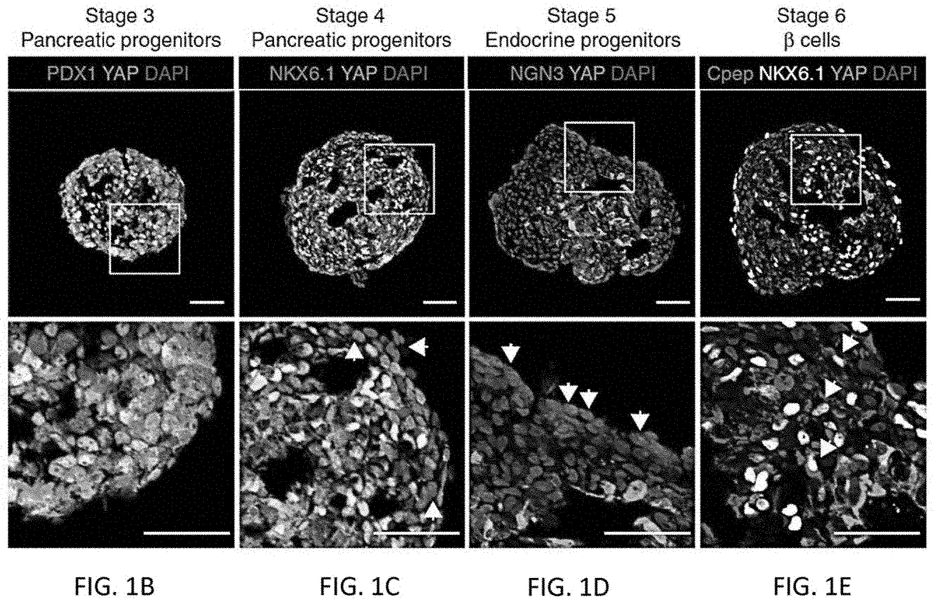

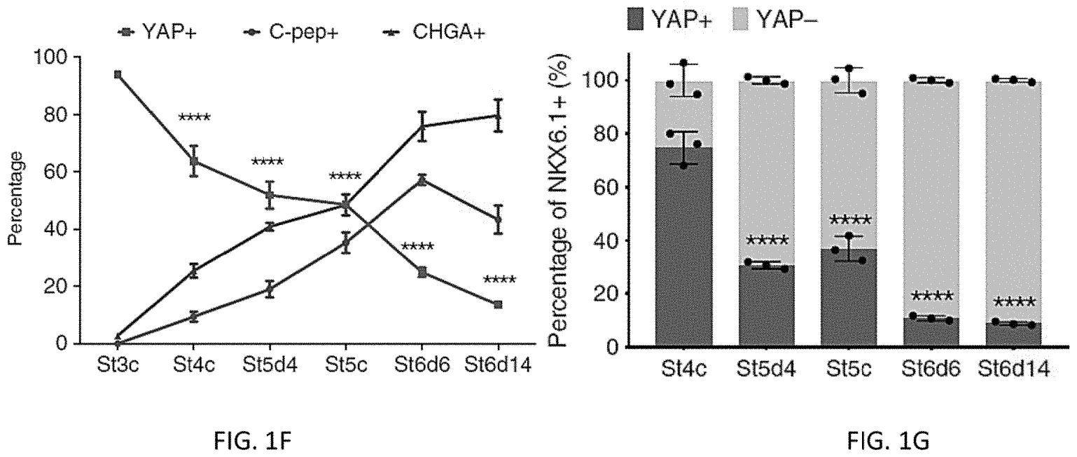

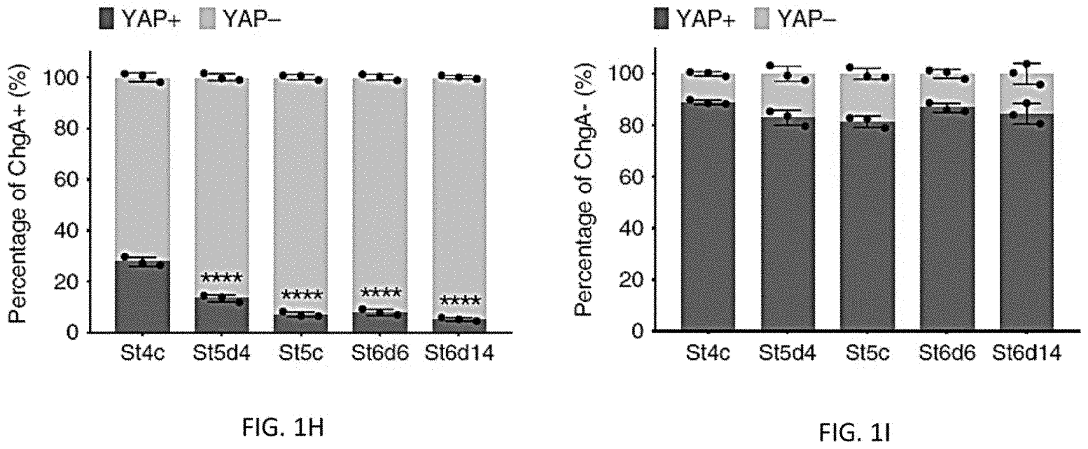

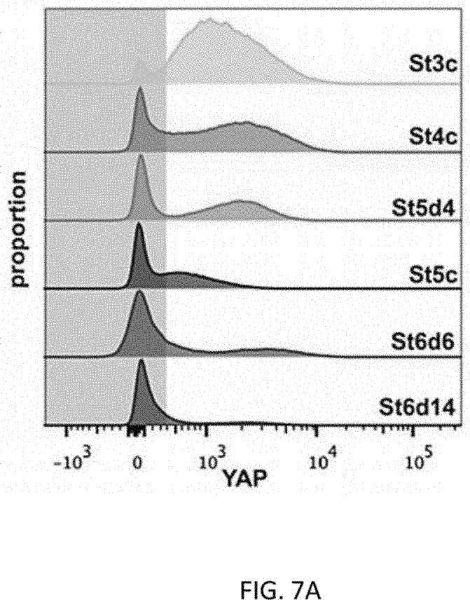

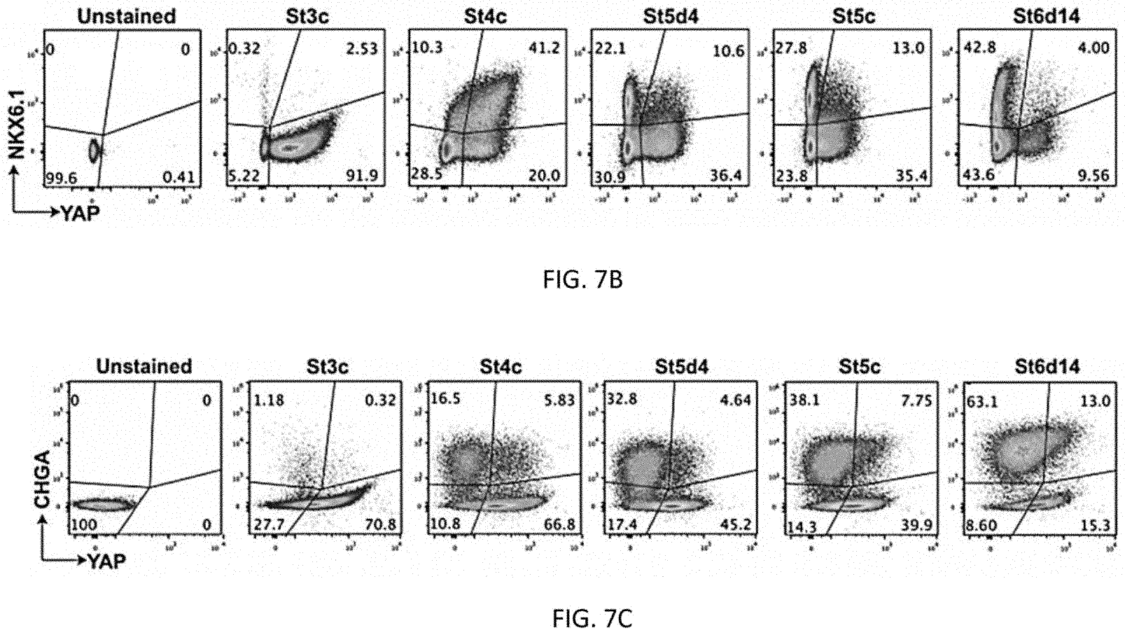

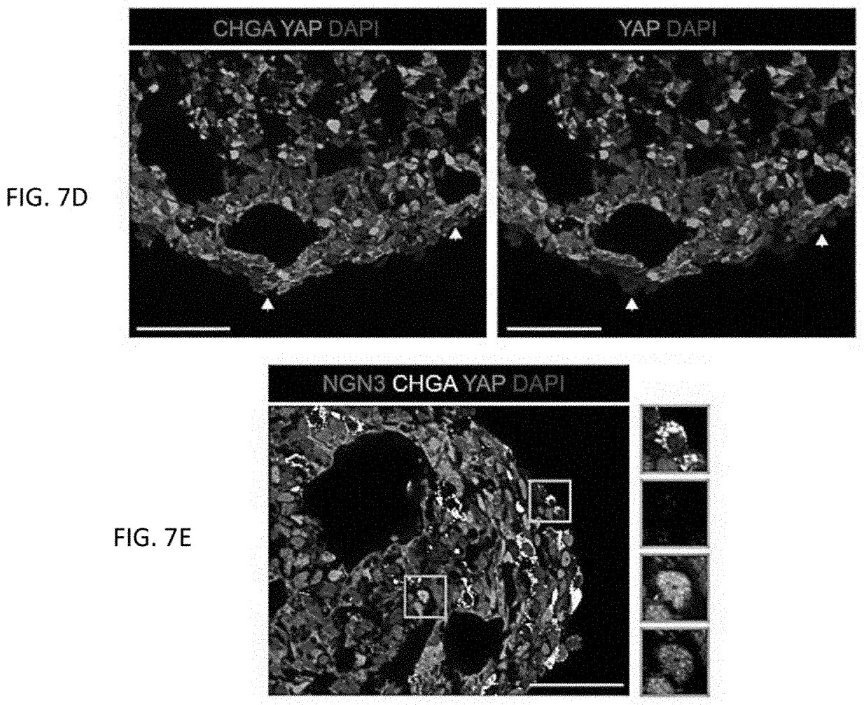

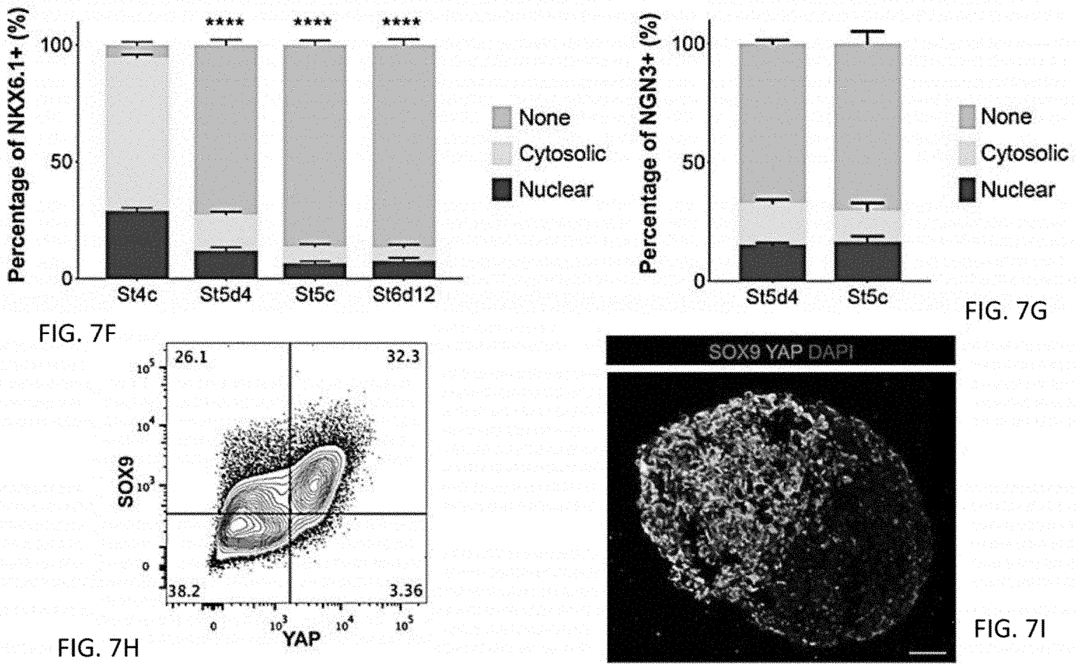

YAP expression was examined during the multistep directed differentiation of hPSCs into β cells as outlined in FIG. 1A [3]. YAP protein expression was observed throughout stages 3-6 (FIGS. 1B-1F and FIGS. 7A-7C), including in PDX1+ early and NKX6.1+ late MPPs at stages 3 and 4 of differentiation, respectively (FIGS. 1B-1C). YAP downregulation begins late in stage 4 NKX6.1+ MPPs and is correlated with the expression of the pan-endocrine marker CHGA (FIGS. 1C, 1F-1G and FIGS. 7A-7D). Although cytoplasmic and nuclear YAP expression is present in NKX6.1+ cells at stage 4, YAP expression in this subpopulation of MPPs further declines as differentiation proceeds into the endocrine lineage (FIG. 1G and FIGS. 7B, 7F).

Because differentiation of MPPs is not synchronous, both early (NGN3+) and late (CHGA+) endocrine progenitors are present at stage 5, day 3 Immunostaining of cell clusters at this time point shows nuclear and cytoplasmic expression of YAP in ˜30% of early endocrine progenitors (NGN3+; FIGS. 1E, 1G), whereas a high proportion of late endocrine cells (CHGA+) have lost YAP expression (FIG. 1H and FIGS. 7C, 7E). Reduced YAP expression in endocrine cells persists upon completion of the directed differentiation protocol: —95% of CHGA+ endocrine and insulin-expressing β cells do not express YAP (FIGS. 1E-1F, 1H and FIG. 7C). At these latter stages of differentiation, YAP expression is largely restricted to non-endocrine cells that co-express the ductal marker SOX9+ (FIG. 1I and FIGS. 7H-7I). In all, YAP is expressed during the progenitor stages and its downregulation correlates with the commitment of MPPs into the endocrine and then beta cell lineages (FIG. 1F).

### YAP is Necessary for the Specification of Late MPPs

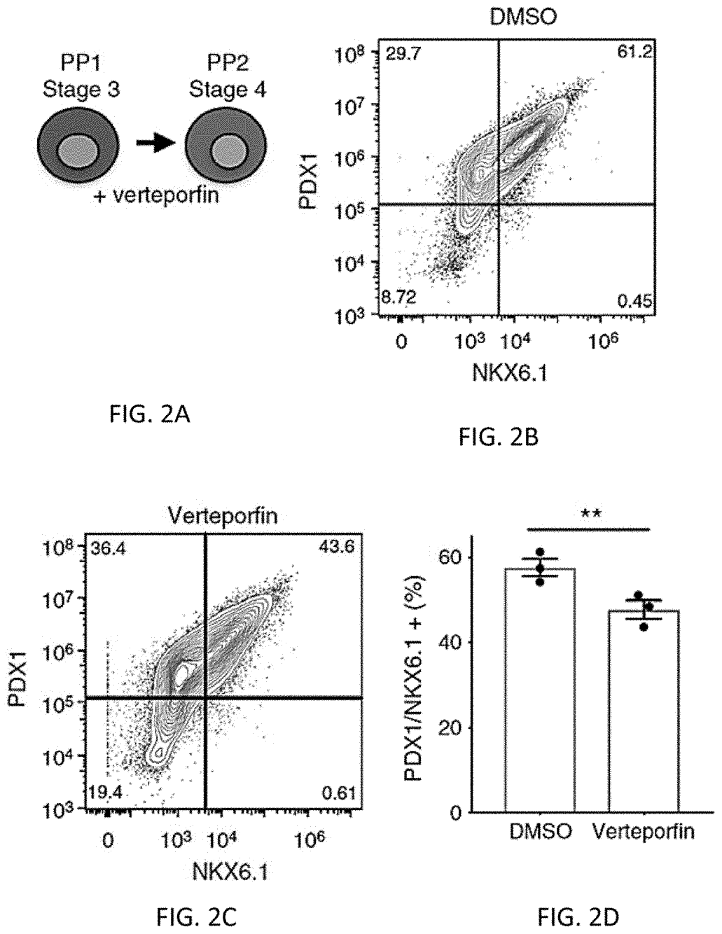

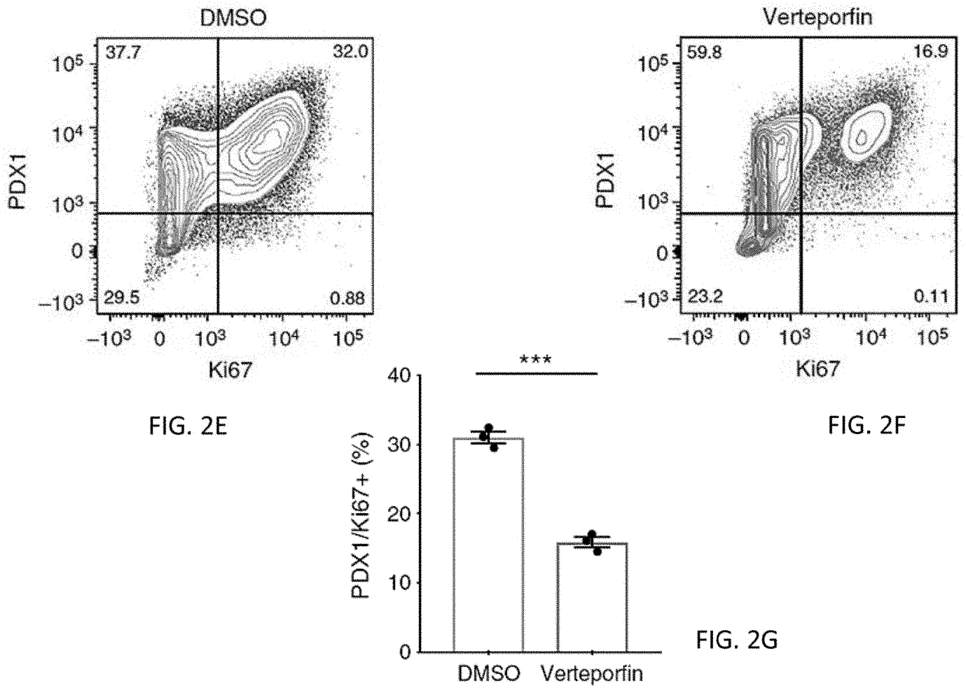

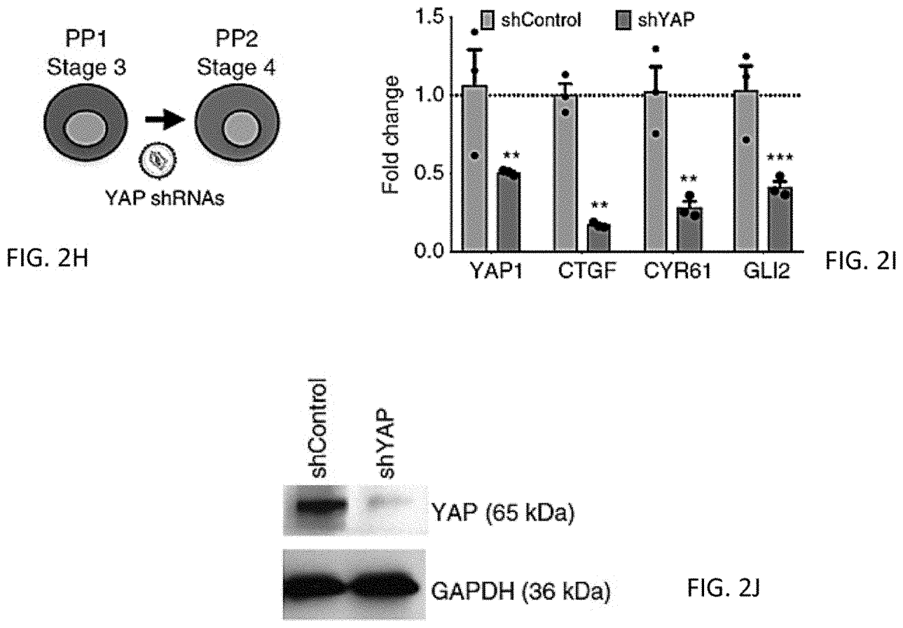

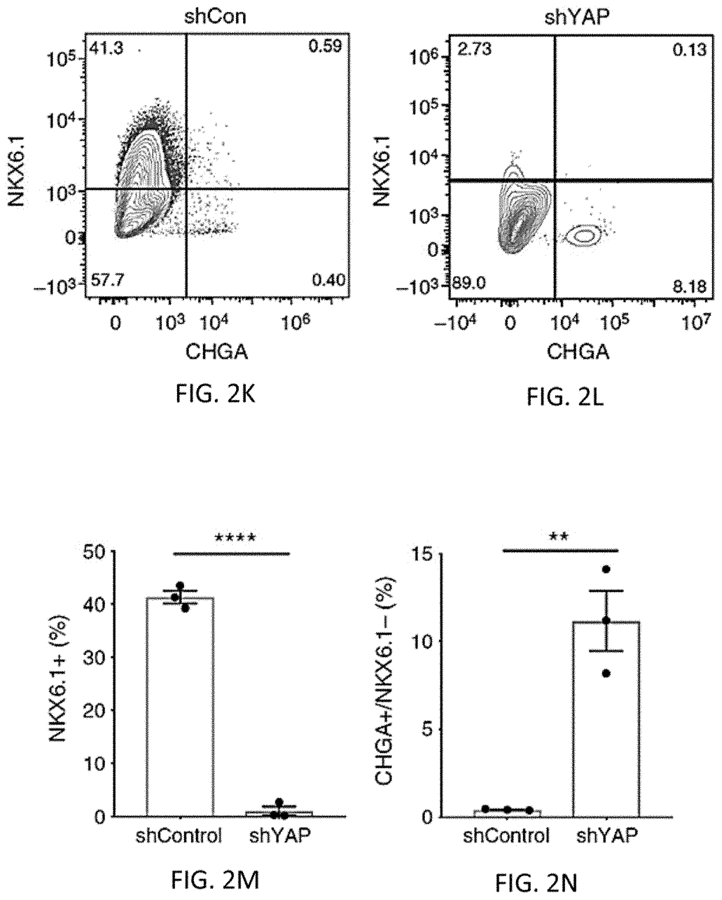

Whether YAP regulates the balance between progenitor self-renewal and differentiation was examined by treating cells with the YAP inhibitor verteporfin. This small molecule inhibits the interaction between YAP and TEAD coactivators and downstream target gene expression [26]. When verteporfin is added during the specification of early PDX1+ to late NKX6.1+ MPPs (FIG. 2A), there is a significant decrease in the number of NKX6.1+ progenitors compared with control differentiations, without any detectable effects on cell viability (FIGS. 2B-2D). The treatment also results in a twofold decrease in the proportion of MPPs coexpressing the proliferation marker Ki67 (FIGS. 2E-2G), consistent with the previous analysis [24]. In agreement with this result, YAP-targeted shRNAs expressed in early pancreatic progenitors during this stage of differentiation (FIG. 2H) results in a twofold downregulation of YAP at the RNA and protein level (FIGS. 2I-2J) and dramatically reduces the proportion of NKX6.1+ progenitors with respect to control differentiations (1.1±0.8 vs. 41.4±1.2%; FIGS. 2K-2M). Concomitantly, a premature differentiation of MPPs into CHGA+/NKX6.1− endocrine cells was observed in cultures of progenitors expressing YAP shRNAs (FIG. 2N). These results are consistent with a role for YAP in promoting the proliferation and expression of NKX6.1+ in MPPs and in restraining their differentiation into the endocrine lineage.

### YAP Inhibition Enhances the Differentiation of β Cells

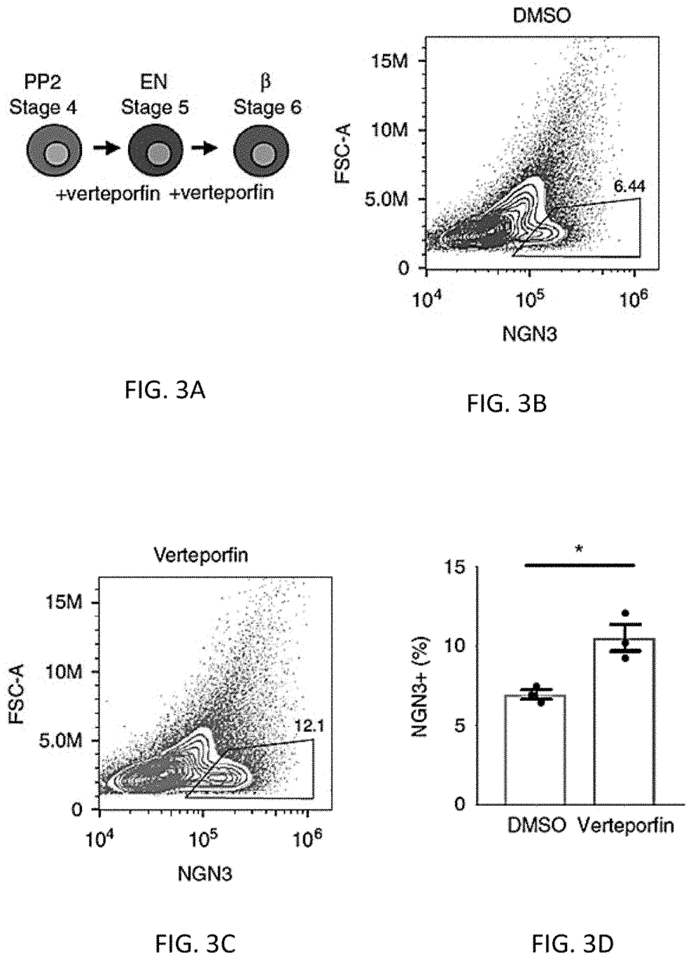

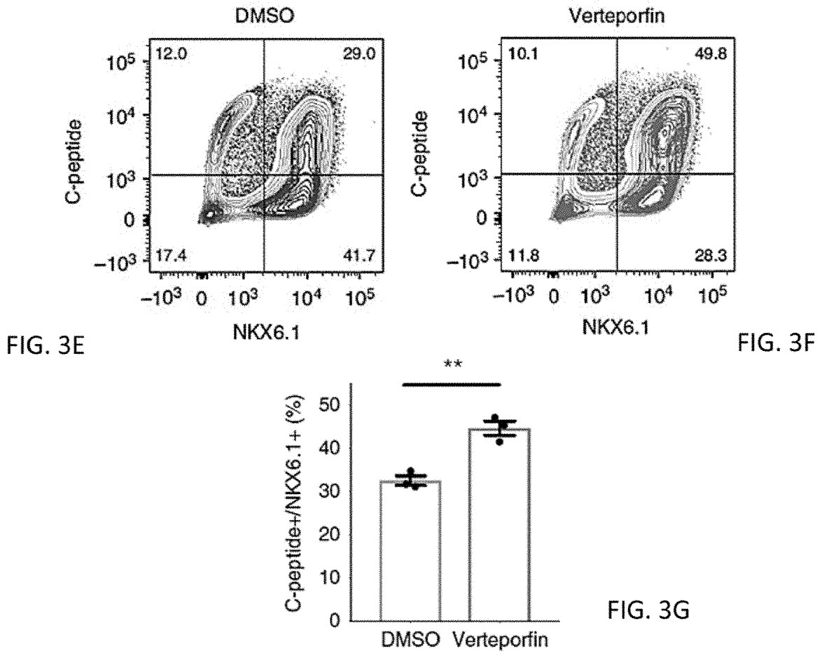

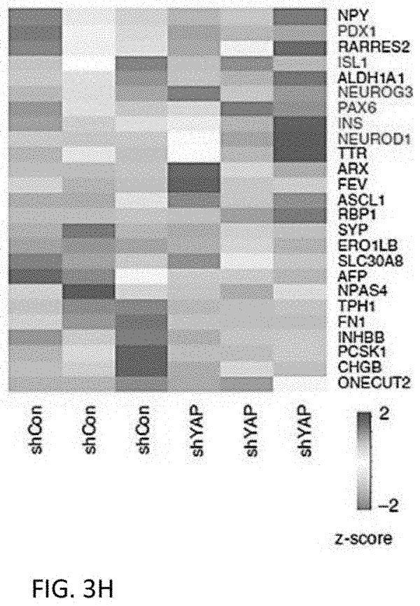

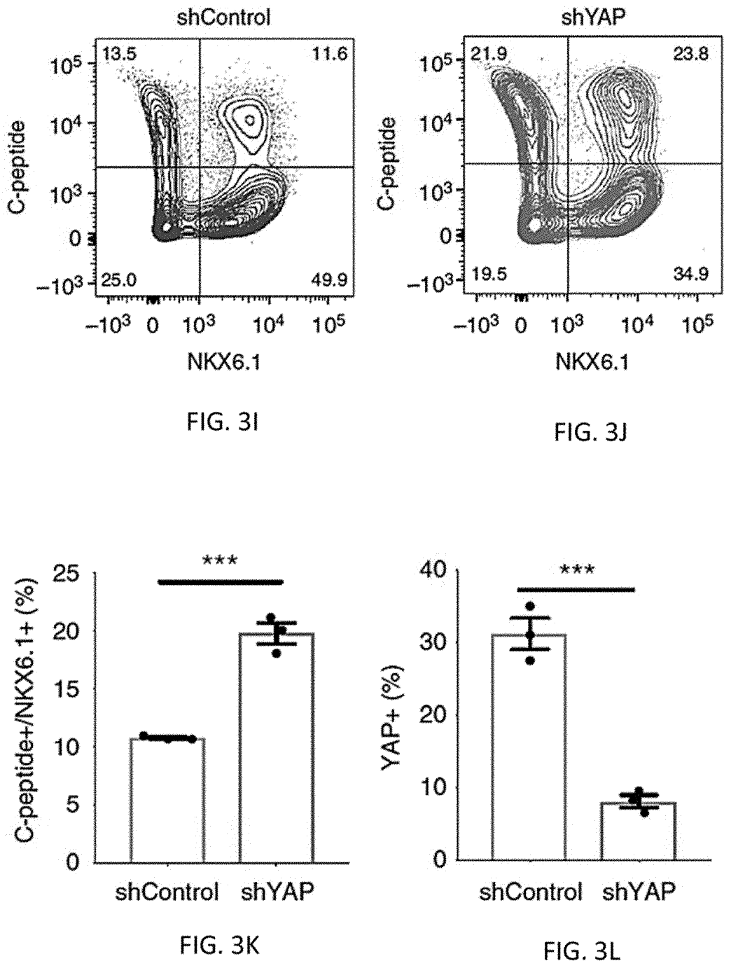

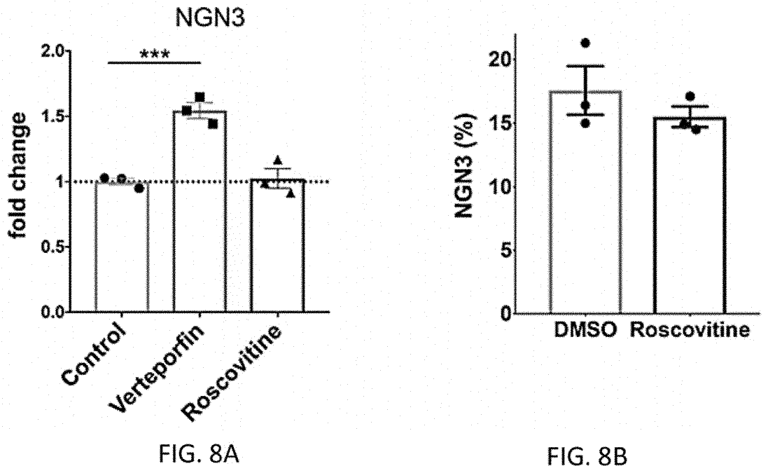

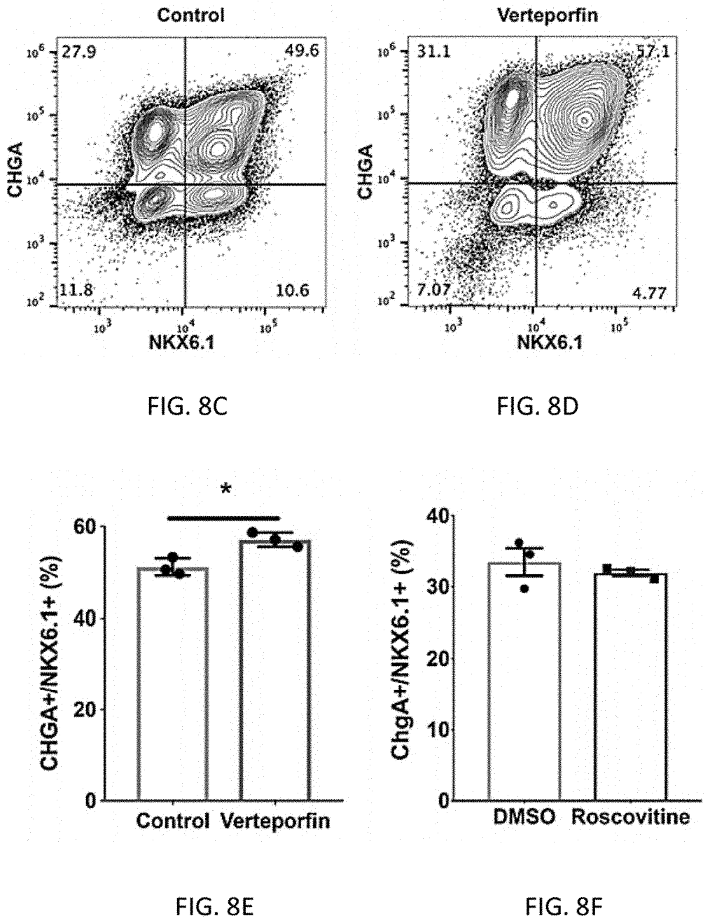

Since YAP activity is correlated with the self-renewal of progenitor cells [20,21] and its downregulation correlates with their commitment to the endocrine lineage (FIGS. 1D-1I and FIGS. 7A-7C), it was hypothesized that YAP downregulation might direct the differentiation of MPPs into the endocrine and β-cell lineages. When YAP was inhibited by verteporfin during endocrine specification at stage 5, MPPs differentiated into NGN3+ endocrine progenitors more efficiently than DMSO controls, as assayed at stage 5, day 4 (FIGS. 3B-3D and FIG. 8A; 12.1±2% verterporfin treatment vs. 6.4±0.52% control). Treatment with roscovitine, a cell-cycle inhibitor, at this stage did not have the same effect (FIGS. 8A-8B), suggesting that blocking proliferation per se does not promote differentiation. The enhanced differentiation of NGN3+ endocrine precursors with verteporfin produces an increase in CHGA+/NKX6-1+ endocrine progenitor cells by the end of stage 5, something not observed with roscovitine treatment (FIGS. 8C-8F, assayed at stage 5, day 7).

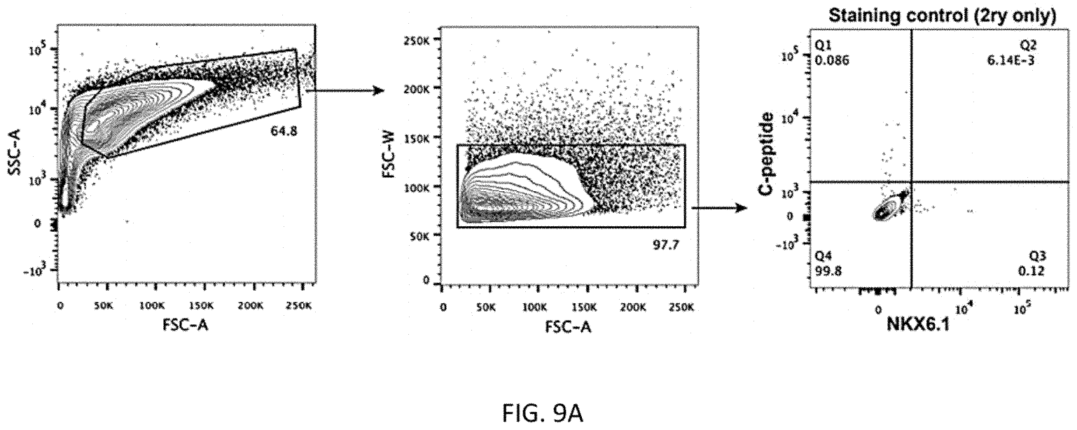

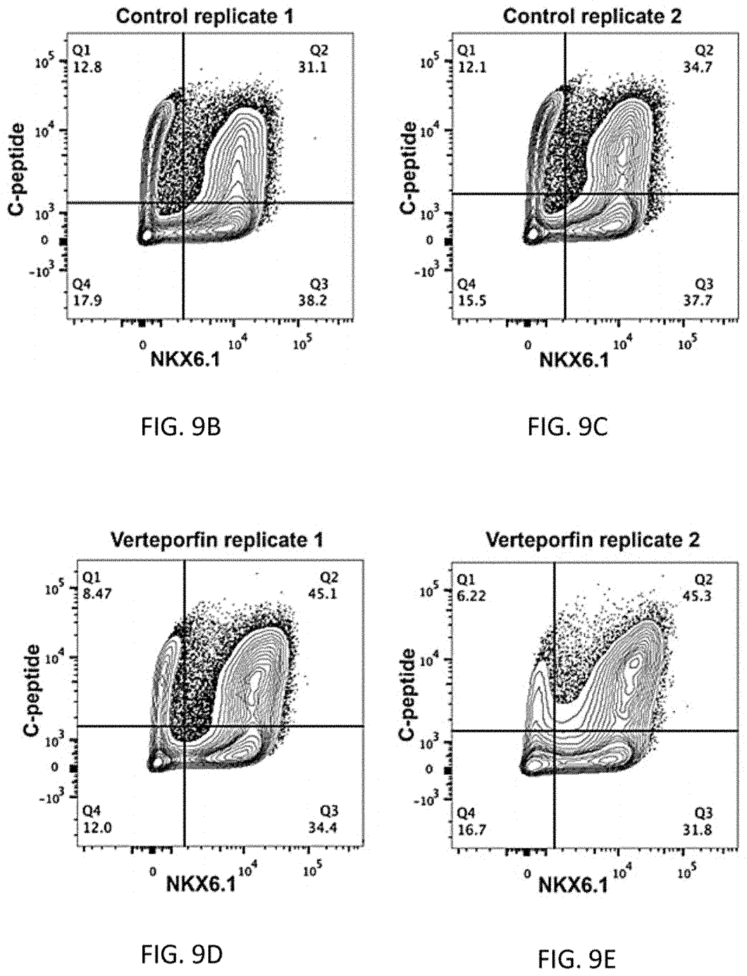

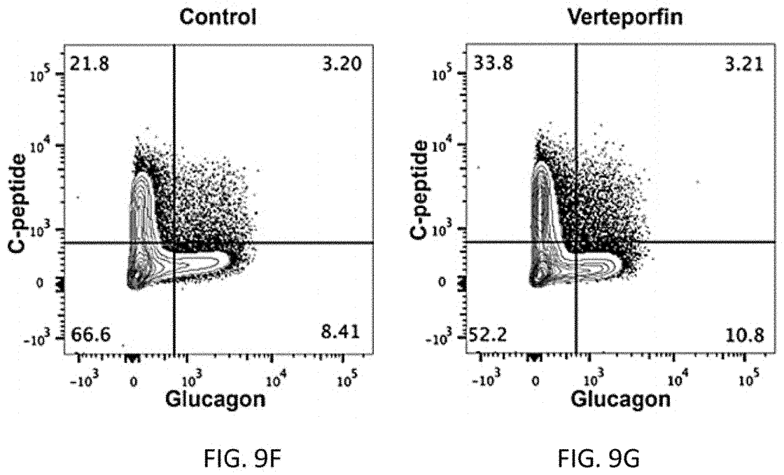

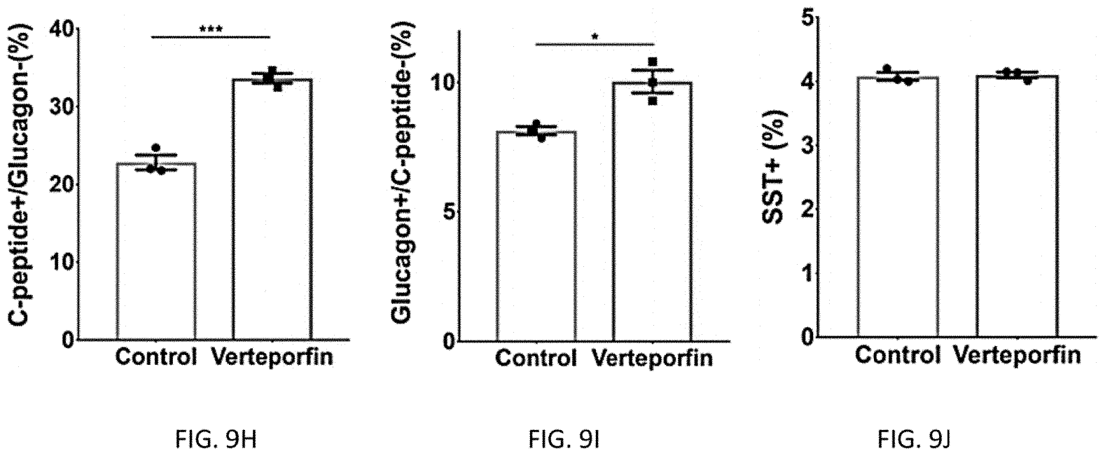

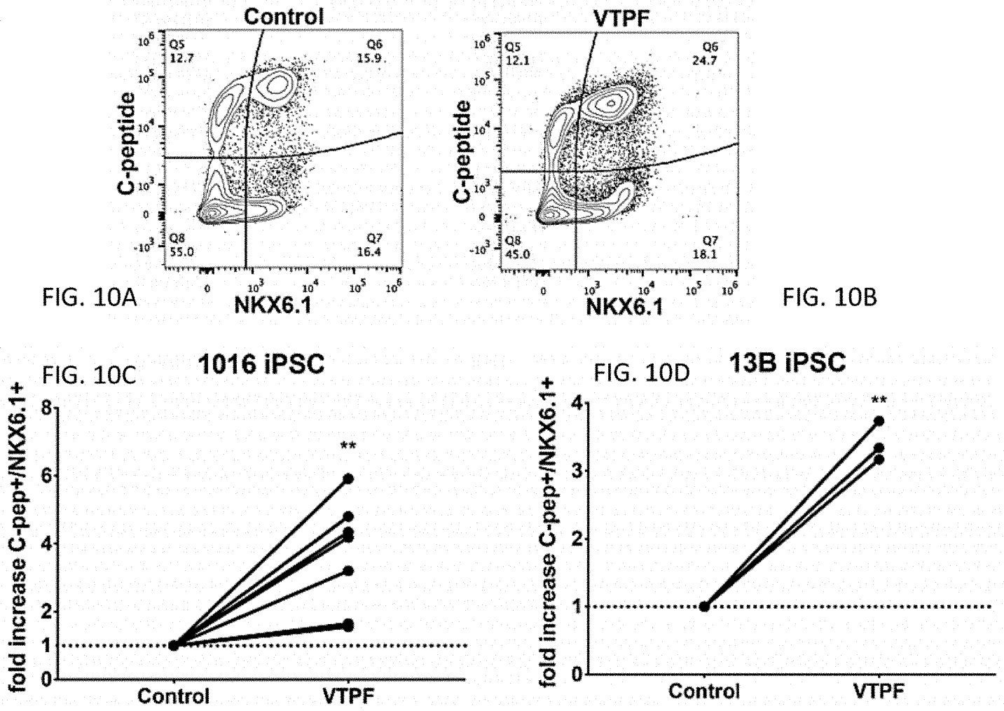

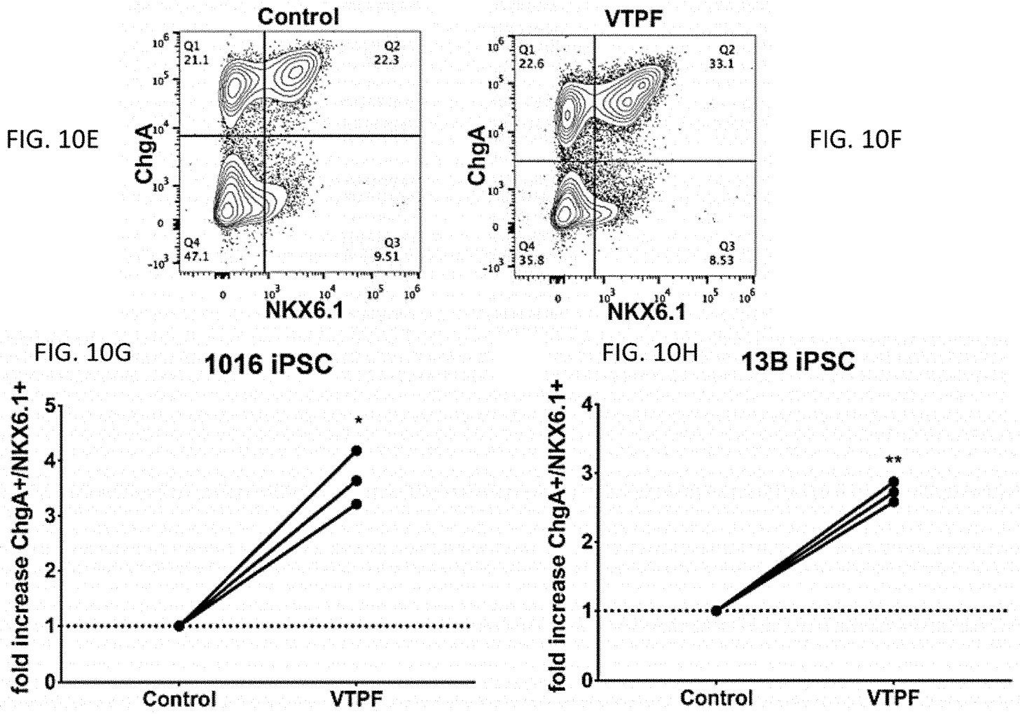

The effect of YAP inhibition was then tested from stage 4 through stage 6 of the β-cell differentiation protocol (FIG. 3A). YAP inhibition by verteporfin during these stages of differentiation leads to a significant increase in the proportion of C-peptide+/NKX6.1+ β cells (FIGS. 3E-3G and FIGS. 9A-9H, 38.6±3.9% verteporfin treatment vs. 27.5±4.2% control). A modest but significant increase in glucagon+/C-peptide-alpha cells, but not somatostatin+ delta cells, is also detected upon YAP inhibition (FIGS. 9F-9I). The effect of verterpofin on SC-endocrine and β-cell differentiation is robust and independent of the genetic background of the hPSC cell line (FIG. 10).

Expression of YAP-targeted shRNAs during endocrine differentiation at stages 5 and 6 produces an increase in NGN3 (NEUROG3), PAX6, and NEUROD1 expression, as well as an increase in insulin (INS) expression (FIG. 3H; assayed at the end of stage 5). Similarly, MPPs expressing YAP-targeted shRNAs differentiated more efficiently into C-peptide+/NKX6.1+β cells than progenitors expressing control shRNAs (FIGS. 3I-3L; assayed at the end of stage 6 differentiation, stage 6, day 14). Thus, YAP inhibition promotes the differentiation of MPPs into the endocrine lineage.

### Sustained Activation of YAP Impairs β-Cell Differentiation

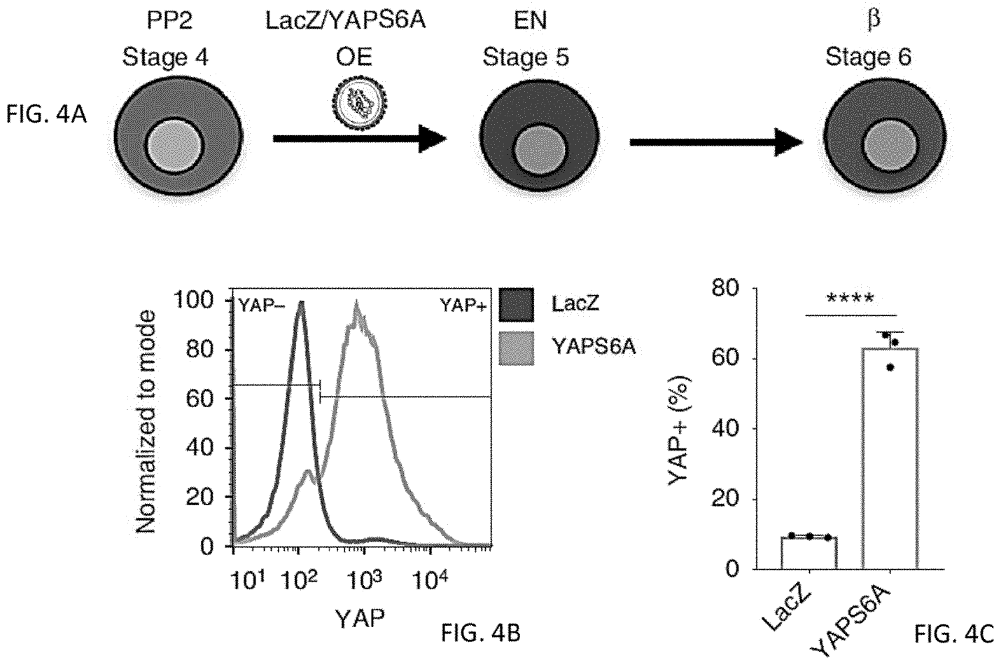

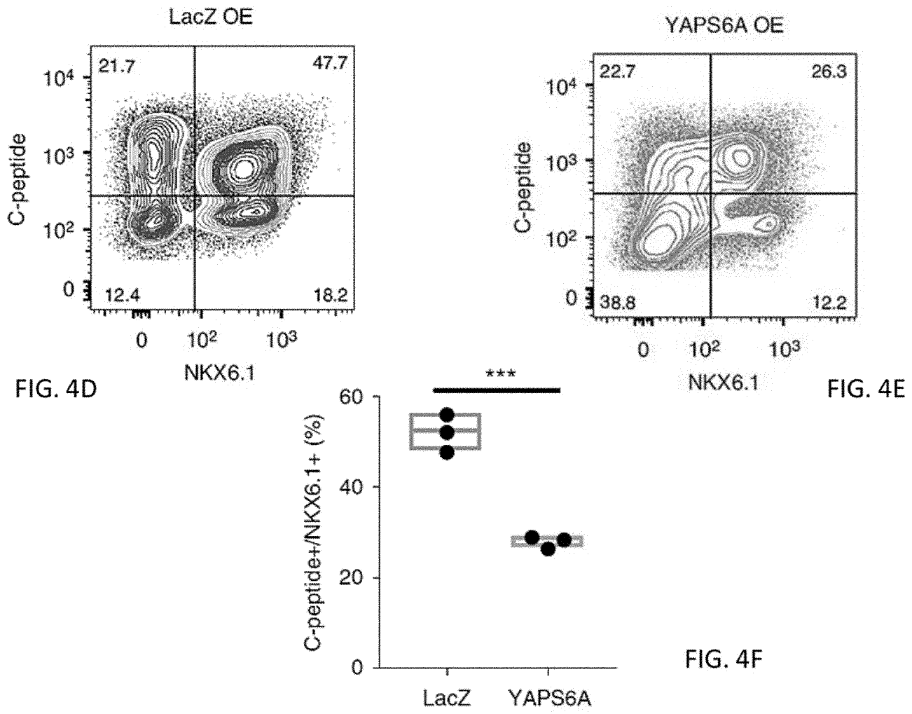

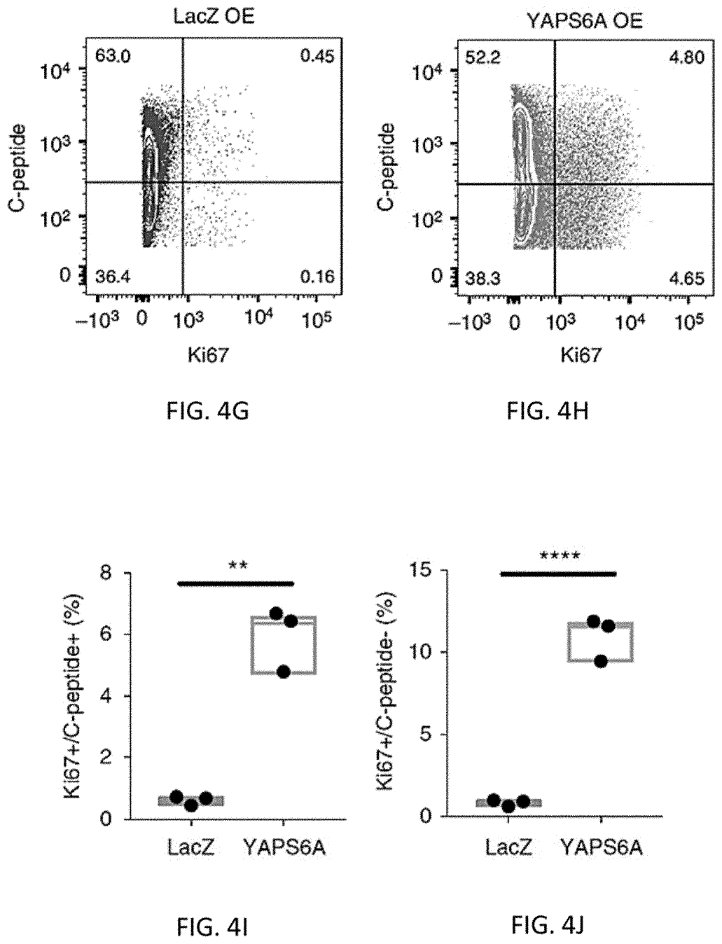

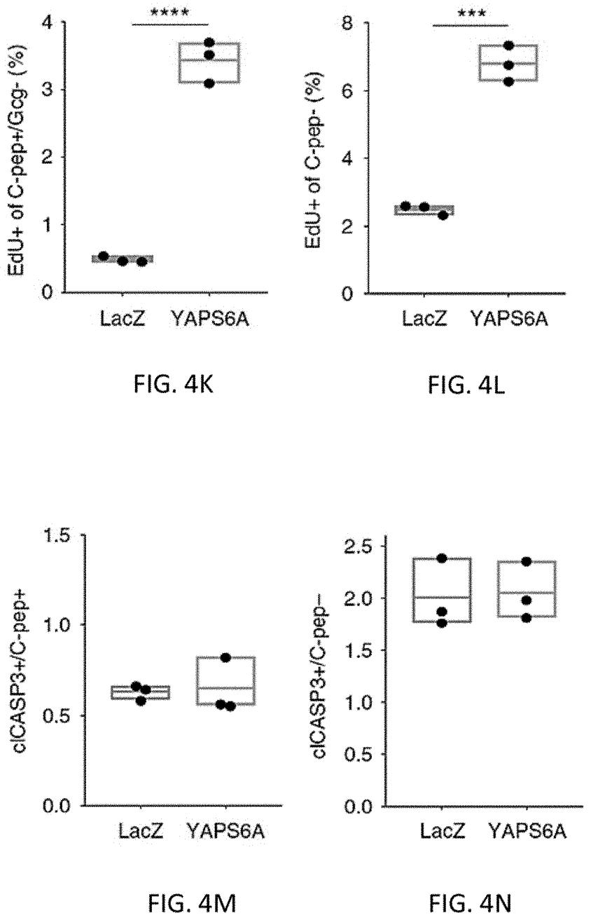

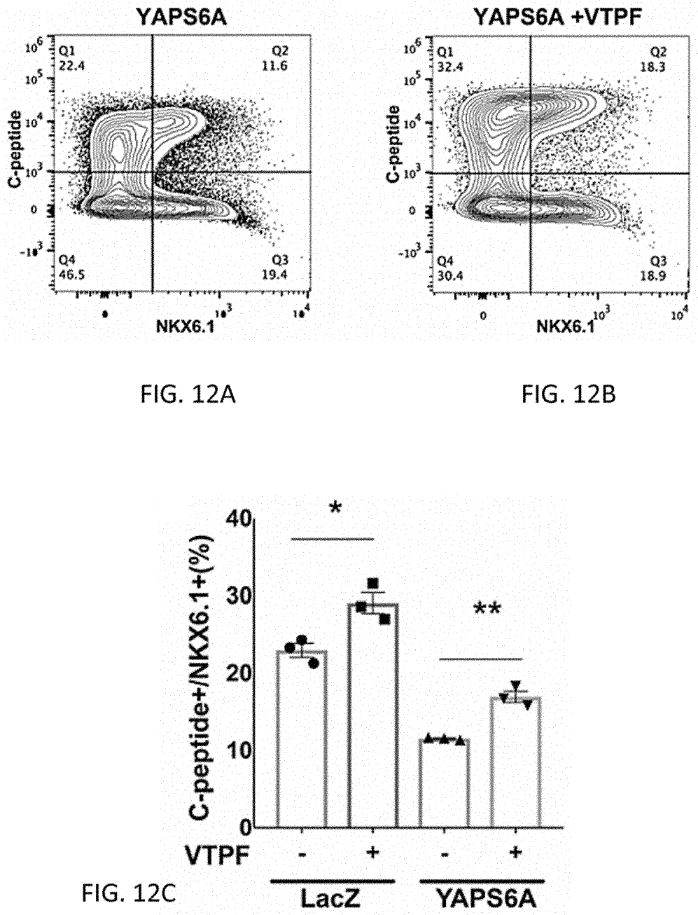

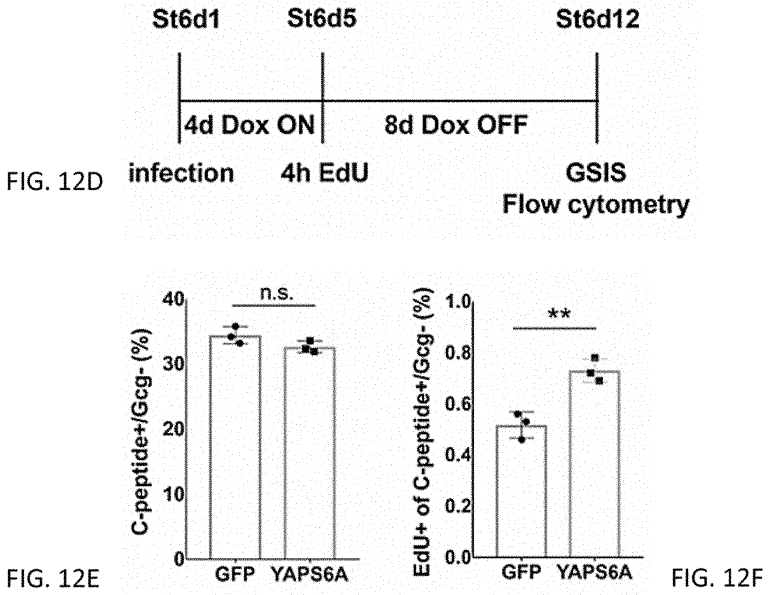

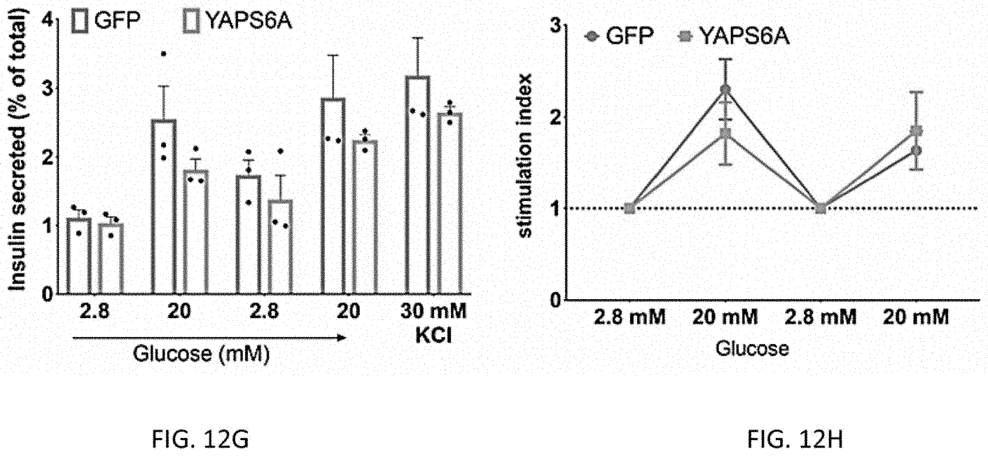

A stabilized form of YAP (YAPS6A) [27] was overexpressed in differentiating MPPs during the last two stages of differentiation (FIG. 4A). Lentiviral overexpression of YAPS6A resulted in a sevenfold increase in the proportion of cells expressing YAP compared with LacZ controls (FIGS. 4B-4C; stage 6, day 14). Consistent with a role of YAP expression in limiting endocrine differentiation, YAPS6A overexpression leads to a significant decrease in the proportion of C-peptide+/NKX6.1+β cells, compared with controls, with no effect on cell apoptosis (FIGS. 4D-4F, 4M, 4N; stage 6, day 14). Verteporfin treatment rescued the impaired endocrine differentiation observed upon YAPS6A overexpression (FIGS. 12A-12C). Levels of proliferation of YAPS6A-overexpressing SC-β cells were quantified and a fivefold increase in the proportion of proliferating cells was detected, compared with controls, as measured by coexpression of the proliferation marker Ki67 with C-peptide and EdU incorporation in SC-β cells (FIGS. 4G-4L). This is consistent with previous reports [28,29]. The increase in proliferation was not specific to C-peptide+ β cells as an increase in the proliferation of non-β cells was observed as well (FIGS. 4J, 4L). These results demonstrate that YAP activity during endocrine differentiation limits the differentiation of MPPs into C-peptide+/NKX6.1+β cells and, instead, promotes continued proliferation.

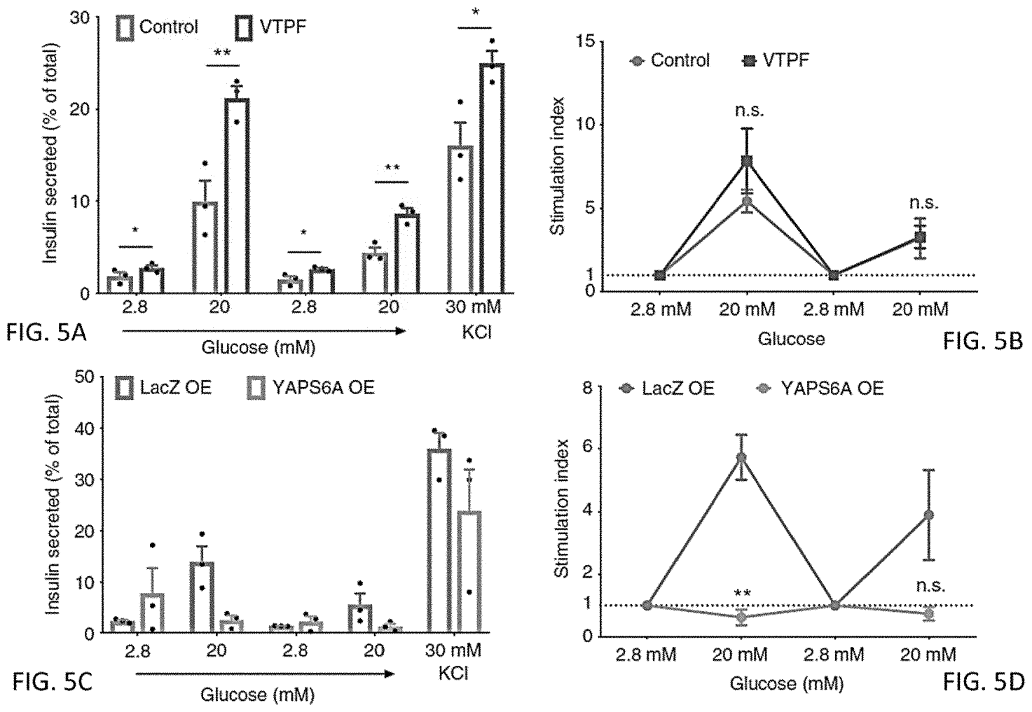

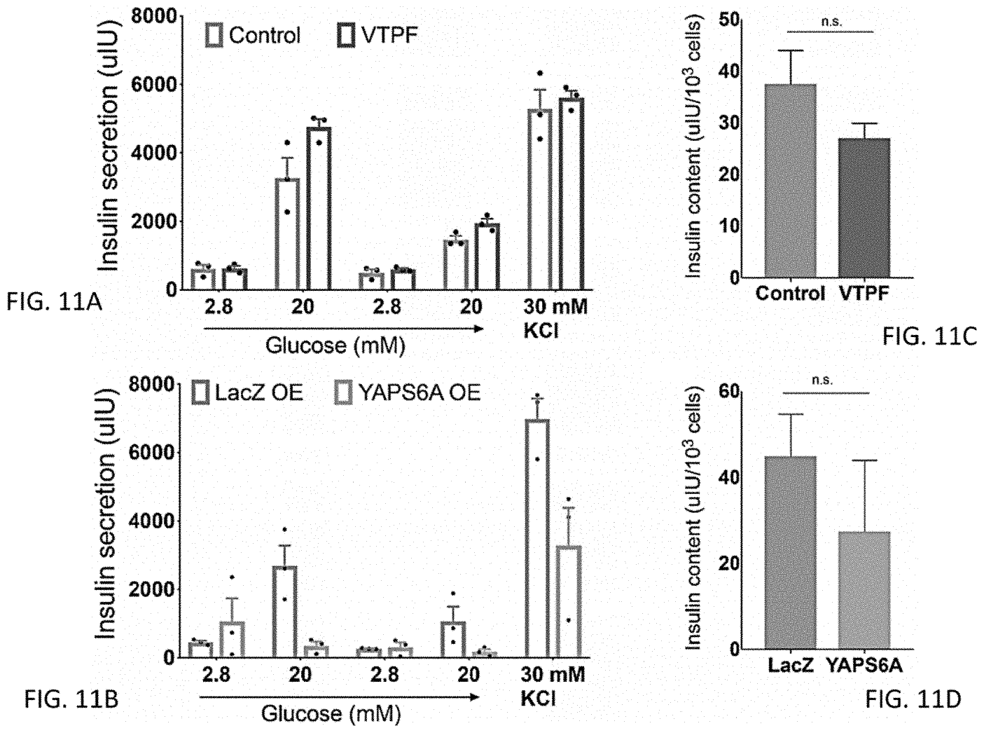

Functional β cells secrete insulin in response to glucose. Endocrine cells supplemented with either verteporfin or DMSO during stages 5 and 6 of differentiation (FIG. 3A) show an increase in insulin secretion at high glucose over low glucose in response to sequential glucose stimulations (FIGS. 5A-5B). Moreover, the levels of insulin secretion per total insulin content at 2.8 mM glucose, 20 mM glucose, and 30 mM KCl were significantly higher in cultures of SC-β cells differentiated with verteporfin (FIG. 5A and FIG. 11). However, there were no statistically significant differences in stimulation indexes between control and verteporfin-treated endocrine cells (FIG. 5B). YAPS6A overexpression during endocrine differentiation (FIG. 4A) hinders the ability of stem cell-derived β cells to secrete insulin in response to glucose (FIGS. 5C-5D). Importantly, YAPS6A-overexpressing β cells secreted insulin in response to KCl stimulation suggestive of defective GSIS in β cells. YAPS6A overexpression leads to a reduction in stimulation indexes compared with LacZ overexpression controls (FIG. 5D). Interestingly, a transient activation of doxycycline-inducible YAPS6A during the first 4 days of stage 6 leads to an increased proliferation of SC-β cells (FIGS. 12D-12F), but does not affect the function of SC-β cells when assayed at stage 6, day 12 (FIGS. 12G-12H). Thus, YAP inhibition may promote the differentiation of SC-β cells that have improved insulin secretion and sustained expression of YAP during endocrine differentiation restricts the differentiation of functional SC-β cells.

### Depletion of Progenitor-Like Cells Upon YAP Inhibition

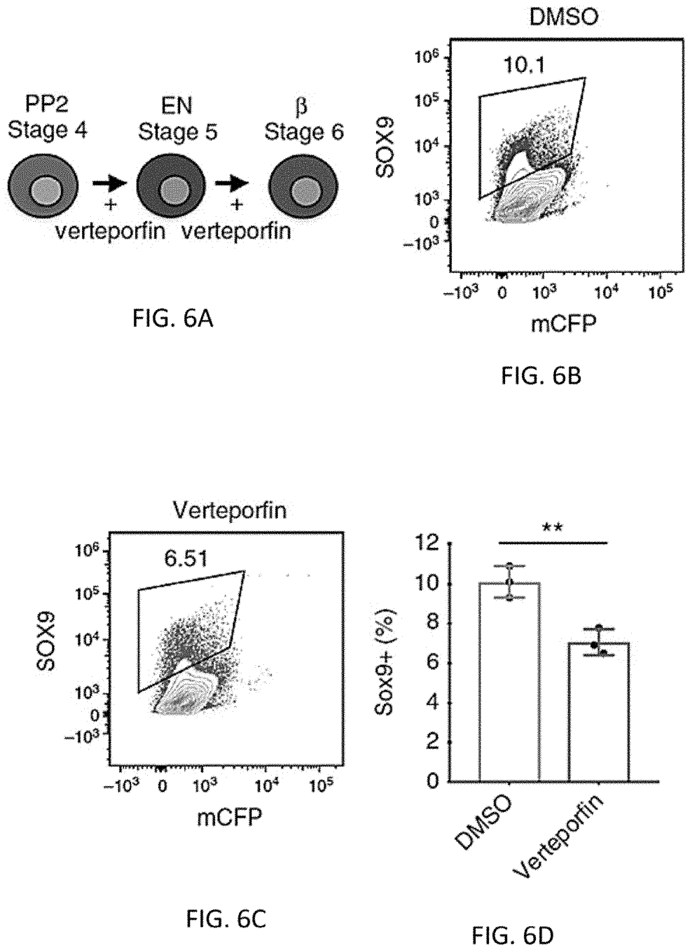

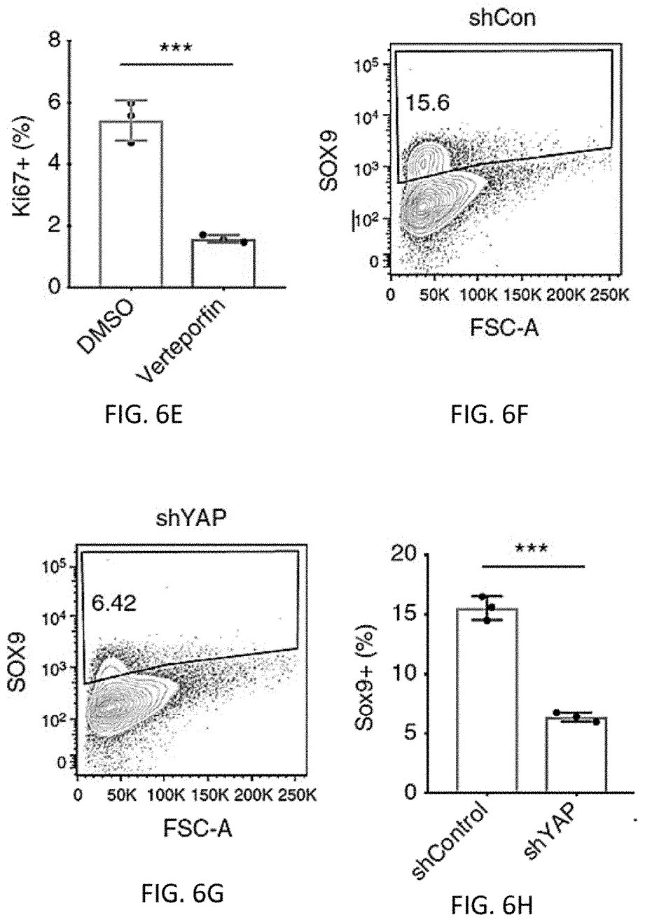

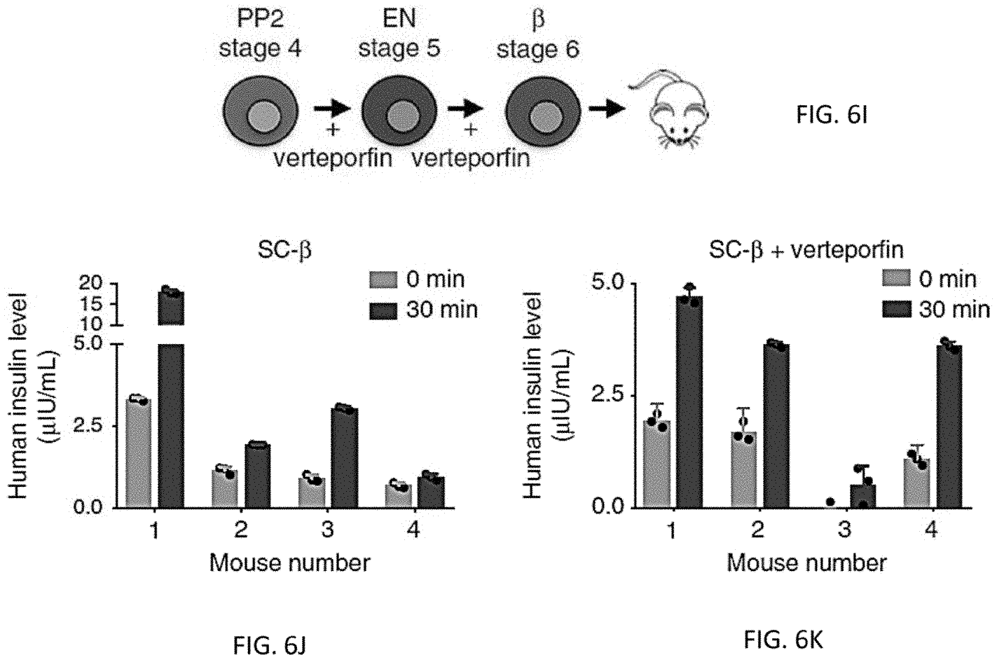

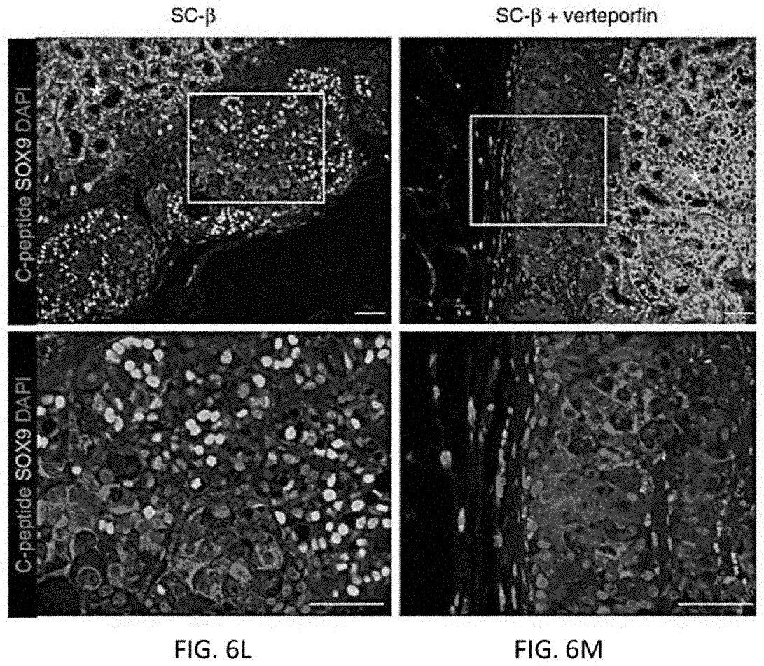

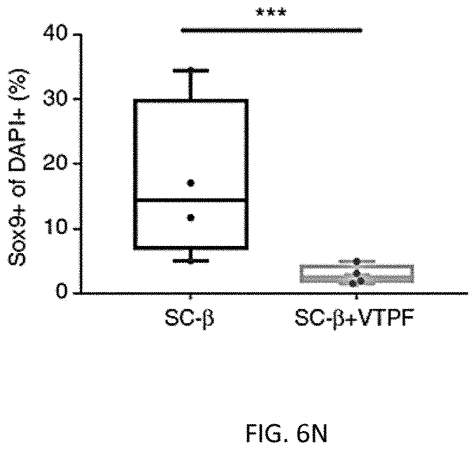

In the adult mouse pancreas, YAP expression is restricted to centroacinar and ductal cells and correlates with increases in levels of cell proliferation [23,30]. In all, 10-15% of the cells at the end of the β cell directed differentiation protocol express Ki67 and the ductal marker SOX9 (FIGS. 6B, 6D-6E, 6H). Given that a portion of SOX9+ ductal-like cells coexpress YAP (FIGS. 7H-7I), it was tested whether YAP inhibition would deplete this subpopulation of cells. Addition of verteporfin during the endocrine and β-cell differentiation stages (FIG. 6A) results in a decrease in the proportion of both SOX9+ ductal-like progenitor cells (FIGS. 6B-6D) and Ki67+ proliferating cells (FIG. 6E). Similarly, expression of YAP shRNAs during these stages leads to a threefold decrease in the proportion of proliferative SOX9+ ductal progenitor cells (FIGS. 6F-6H).

Following transplantation, Sox9+ progenitor cells may significantly expand and form unwanted cells [19]. To test whether YAP inhibition depletes these proliferative cells in vivo, SC-β clusters were transplanted into the kidney capsule of immunocompromised mice [3]. Transplants of both control SC-β cells and cells differentiated in the presence of verteporfin (FIG. 6I) displayed robust in vivo glucose-stimulated human insulin secretion (FIGS. 6J-6K). At 12-week post transplantation, the proportion of SOX9+ cells in the kidney grafts and the expression of this marker was detected in ˜17% of the cells in control grafts (FIGS. 6L-6N). Transplants of SC-β cells differentiated in the presence of verterporfin displayed a reduction in the proportion of SOX9+ cells with an average of 3.1% in the grafts at this time point (FIGS. 6M-6N). The data suggest that YAP inhibition can partially deplete ductal-like and proliferative cells that may expand following transplantation.

### Discussion

The generation of functional SC-β cells from hPSCs relies on the modulation of signaling effectors, most of which were known to control in vivo pancreas organogenesis [3-5]. Efforts to elucidate novel cell fate determinants will not only shed light into this process but will also result in improved directed differentiation protocols for cell replacement therapies. Here YAP is identified, one of the effectors of the Hippo signaling pathway, as a factor involved in progenitor specification and differentiation into functional pancreatic endocrine cells. The regulation of YAP follows a developmental logic whereby its activity promotes the expansion of MPPs and its downregulation corresponds to their differentiation into endocrine progenitors and SC-β cells.

The Hippo signaling pathway controls organ size by coordinating progenitor proliferation and differentiation [21,31-33]. Mice with a pancreas-specific deletion of the upstream regulators MST1/2 displayed extensive acinar-to-ductal metaplasia and acute pancreatitis [22,23]. Sustained activation of YAP during pancreas development, or postnatally, resulted in a disrupted islet architecture and an undeveloped endocrine compartment [22,23,25]. However, dissecting lineage-specific dependencies on YAP activity during in vivo pancreas development is confounded by the extensive organ-wide tissue disarray [22,23]. Exploiting the in vitro differentiation of MPPs, the present study uncovers a role for YAP in limiting the differentiation of human endocrine cells by maintaining pancreatic progenitor identity and self-renewal. In agreement with this study, Cebola et al. showed that YAP and TEAD coactivators are core components of cis-regulatory modules of transcription factors that establish the multipotency and expansion of pancreatic progenitors, including SOX9, NKX6.1, GATA4, GATA6, HHEX, and FOXA2, among others [24]. Consistent with this data, other studies have shown that genetic downregulation or pharmacological inhibition of YAP impairs pancreatic progenitor proliferation [24,34].

MPPs undergo a cell-cycle lengthening and arrest during endocrine induction [8,11-13]. Cell cycle-dependent regulation of NGN3, an indispensable factor for pancreatic endocrine differentiation [7-10], further drives the specification of progenitors into the endocrine lineage [11,12]. However, the molecular underpinnings controlling the cell cycle-dependent specification into pancreatic endocrine lineages remain elusive. The present study lends support for a salient role for YAP in establishing whether a progenitor cell undergoes proliferation or differentiates, similar to the cell cycle-dependent differentiation models put forward for neural differentiation [35,36]. It is also shown that downregulated activity of YAP, either chemically or genetically, enhances the generation of endocrine cells and SC-β cells. Part of this process may be the downregulation of SOX9 upon YAP inhibition. SOX9 is essential for the maintenance of mitotically active MPPs and ductal differentiation during pancreas organogenesis [37,38] and its expression is controlled by YAP in other cell types [32,39]. As most YAP+ cells at the end of the directed differentiation protocol coexpress SOX9, it is further posited that a failure to induce YAP downregulation in MPPs may limit their differentiation into endocrine lineages and contribute to the cellular heterogeneity observed during in vitro differentiation of SC-β cells [3,4]. Further support for this interpretation comes from a recent study that uncovered a mechanotransduction-dependent role of YAP in ductal and endocrine lineage bifurcation [25].

In all, it is proposed that a dual modulation of YAP activity can improve the directed differentiation into the β-cell lineage. Evidence is provided for the utility of the YAP inhibitor verteporfin as a potent inducer of endocrine differentiation and progenitor depletion in vitro. In addition, it is hypothesized that molecules that enhance YAP activity during the specification of MPPs may promote the expression of NKX6.1 in this population of progenitors, as the data link YAP activity to its expression, critical for the formation of monohormonal β cells at later stages of differentiation [3,4,14,15]. Given the known role of Hippo signaling and YAP during organ development [20], this approach may be applicable to other directed differentiation models that guide the differentiation of hPSCs into post-mitotic cells.

### Materials and Methods

Cell Culture and Differentiation of hPSCs

hPSCs were maintained in mTeSR1 (Stem Cell Technologies) in 500-mL spinner flaks on a stir plate (Chemglass) set to 70 rpm in a 37° C. incubator, 5% CO₂, and 100% humidity. All the experiments were carried out using the human embryonic stem cell line HUES8 and the induced pluripotent stem cell lines (iPSC) 1016 and 13B [3,40]. Cell lines were obtained from the Human Embryonic Stem Cell Facility and iPS Core Facility of the Harvard Stem Cell Institute, and University of Massachusetts Medical School.

Differentiations into SC-β cells were performed following a protocol described in [3] and as follows: HUES8 or 1016 cells were seeded at 6×10⁵ cells/mL in mTeSR1 media and 10 μm Y27632 (Sigma-Aldrich). The media was changed 48 h later and the differentiations were started 72 h after the cells were seeded. The media changes were as follows:

Stage 1 DE: Day 1: S1+100 ng/mL ActivinA (R&D Systems)+3 μM Chir99021 (Stemgent). Day 2: S1+100 ng/mL ActivinA.

Stage 2 GTE: Days 4 and 6: S2+50 ng/mL KGF (Peprotech).

Stage 3 PP1: Days 7 and 8: S3+50 ng/mL KGF+0.25 μM Sant1 (Sigma)+2 μM RA (Sigma)+200 nM LDN193189 (only Day 7) (Sigma)+500 nM PdBU (EMD Millipore).

Stage 4 PP2: Days 9, 11, and 13: S3+50 ng/mL KGF+0.25 μM Sant1+100 nM RA+10 μm Y27632+5 ng/mL ActivinA.

Stage 5 EN: Days 14 and 16: S5+0.25 μM Sant1+100 nM RA+1 μM XXI (EMD Millipore)+10 μM Alk5i II (Axxora)+1 μM T3 (EMD Millipore)+20 ng/mL Betacellulin (Thermo Fisher Scientific). Days 18, 20: S5+25 nM RA+1 μM XXI+10 μM Alk5i II+1 μM T3+20 ng/mL Betacellulin.

Stage 6 (3: Days 21-35 (change every other day): S3 media. In the final stage, cells were analyzed between 28 and 35 days of the protocol.

Control and experimental samples were collected from differentiations performed in parallel with hPSC cells obtained from the same maintenance batch. Treatments with 0.35 μM verteporfin (Sigma-Aldrich), 10 μM roscovitine (Sellekchem), or DMSO (Sigma-Aldrich) were performed at the last stages of the differentiation including stage 4 (days 9-14), stage 5 (days 14-18), and stage 6 (day 18-25, first 7 days) in ultra-low attachment six-well plates (Corning).

### Lentivirus Production and Infection of Cells

Lentiviral particles were produced by transfecting 293 T cells (Takara Bio) with the packaging vectors pHDM-vsvg, pHDM-tat, pHDM-rev, and pHDM-gag/pol along with lentiviral backbone vectors using the TranslT-293 transfection reagent (Mirus). The lentiviral vectors used in this study were the following: YAP1(S6A)-pLX304 (addgene #42562), LacZ-pLX304 (addgene #42560), YAPS6A-pLIX403, GFP-pLIX403, 29-mer scrambled shRNA in pGFP-C-shLenti (Origene), and four unique 29mer YAP shRNAs in pGFP-C-shLenti (Origene). Lentiviral particles were concentrated 48 h and 72 h post transfection using the PEG-IT virus precipitation reagent (Fisher Scientific) overnight at 4° C. followed by centrifugation at 1500 g for 30 min at 4° C. and stored at −80° C.

For infection, cell clusters collected from spinner flask suspension cultures were dissociated in Accutase (StemCell Technologies) for 10 min, followed by mechanical dissociation and centrifugation at 230 g for 5 min at room temperature (RT). Cell pellets were resuspended at a density of 2.5 million cells/mL in the stage-matched medium with polybrene reagent (Santa Cruz) at 8 μg/mL. Single-cell suspensions were combined with concentrated lentiviral particles and plated on ultra-low attachment six-well plates on a rocker plate set at 70 rpm in a 37° C. incubator, 5% CO₂, and 100% humidity. Feeding schedule was followed depending on the stage of differentiation as described above. For the transient induction of GFP and YAPS6A expression, infected cells were treated with 2 μg/mL doxycycline (Sigma-Aldrich), as described here.

### Immunohistochemistry

Cell clusters were collected at the end of each of the differentiation stages and fixed with 4% paraformaldehyde for 1 h at RT, washed in 30% sucrose, embedded in OCT compound (Tissue-Tek) and sectioned for histological analysis. Clusters were blocked with PBS+ 0.1% Triton X-100 (VWR)+5% donkey serum (Jackson Immunoresearch) for 30 min at RT, incubated with primary antibodies overnight at 4° C., washed, and incubated with secondary antibodies for 1 h at RT. Stained clusters were washed and mounted in Flouromount-G (Invitrogen), covered with coverslips, and sealed with nail polish. Images were taken using a Zeis Axio Imager.Z2 and Apotome.2. Image analysis was performed with ImageJ. To estimate the relative proportion of cells in the graft, four tissue sections from different areas in the graft were imaged, and the relative proportions of relevant cell types were quantified as a percent of cells positive for the relevant marker of the total number DAPI nuclei. The antibodies used in the study were the following: rabbit anti-YAP (1:100, Cell Signaling Technology; 14074 S), mouse anti-YAP (1:100, Abnova; 89106308), rabbit anti-SOX9 (1:100, Cell Marque; AC-0284RUO), rat anti-C-peptide (1:200, Developmental Studies Hybridoma Bank; GN-ID4), mouse anti-NKX6.1 (1:100, Developmental Studies Hybridoma Bank; F55A12-supernatant), rabbit anti-Ki67 (1:100, Abcam; ab16667), sheep anti-NGN3 (1:50, R&D systems; AF3444), goat anti-PDX1 (1:100, R&D systems; AF2419), rabbit anti-CHGA (1:200, Novus Biologicals; NB120-15160), mouse anti-CHGA (1:200, Santa Cruz; sc-393941), mouse anti-glucagon (1:200, Abcam; ab82270), mouse anti-somatostatin (1:200, Santa Cruz; sc-55565), rabbit anti-cleaved Caspase-3 (1:100, Cell Signaling; 9661) and mouse anti-PCNA (1:100, Millipore; NA03).

### Flow Cytometry

Differentiated cell clusters were dispersed into a single-cell suspension with TrypLE (Life Technologies) at RT, fixed with 4% paraformaldehyde at 4° C., washed three times in PBS+0.2% bovine serum albumin (Millipore)+0.1% saponin (Sigma), blocked for 30 min at 4° C. in PBS+5% donkey serum+0.1% saponin, incubated with primary antibodies overnight at 4° C., washed, and incubated with secondary antibodies for 1 h at RT. Stained, fixed cells were filtered through a 40-μm nylon mesh into flow cytometry tubes (BD Falcon) and were analyzed for relevant stage-specific marker expression using LSR II flow cytometers (BD Biosciences) and FlowJo for the data analysis. Samples stained with secondary antibodies only were used to gate on cells that are negative and accurately identified cells expressing the markers included in the analysis. For shRNA-infected samples, only infected GFP+ cells were included in the analysis (approximately in 30-40% of all cells). To quantify incorporation of EdU and estimate levels of proliferation, cells pulsed with 10 μM EdU for 4 h were dissociated as described above unless stated otherwise. The Click-IT EdU flow cytometry kit (Invitrogen) was used to detect EdU incorporation following the manufacturer's protocol with minor modifications.

### Glucose-Stimulated Insulin Secretion (GSIS)

Approximately 1×10⁶ SC-β cells in clusters were washed with Krebs buffer and incubated in low (2.8 mM) glucose Krebs in cell culture inserts (Millicell) to starve cells and remove residual insulin at 37° C. for 1 h. Clusters were washed and incubated in low glucose Krebs for 1 h and the supernantant was collected. They were then transferred to high (20 mM) glucose, incubated for 1 h and the supernatant was collected. For sequential GSIS challenges, this sequence of low- and high-glucose stimulations was repeated twice. At the end, clusters were incubated in 2.8 mM glucose+30 mM KCl Krebs (depolarization challenge) for 1 h and the supernatant was collected. Clusters were dispersed into single cells using TrypLE and cell number was estimated by a Vi-Cell counter (Beckman Coulter). Insulin concentration was determined for supernatant samples using the Human Ultrasensitive Insulin ELISA (ALPCO Diagnostics). Protein extraction was performed with the M-PER extraction reagent (Thermo Scientific), and insulin content was measured for each sample using the human Ultrasensitive Insulin ELISA kit. Insulin secretion levels were normalized to total insulin content for each sample. Stimulation indexes were calculated as a ratio of insulin secretion at high glucose (20 mM) relative to the basal secretion (2.8 mM glucose).

### Mouse Transplantation Analysis

Dissociated SC-β cells were transplanted into the kidney capsule of immunodeficient SCID-beige mice (Jackson Laboratory), aged 8-10 weeks [3]. In total, 5×10⁶ differentiated cells (per animal) collected at the end of the SC-β cell differentiation protocol were dispersed using Accutase and resuspended in 200 μL of the RPMI1640 medium and kept on ice for 5-10 min before loading into a catheter for cell delivery under the kidney capsule. Mice were first anesthetized with 0.5 mL/25 g 1.25% avertin/body weight, and the left ventral site was shaved and betadine and alcohol was applied to clean the incision site. A 1-cm incision was performed to expose the kidney for the insertion of the catheter needle and injection of the cells. The abdominal cavity was closed with PDS absorbable sutures (POLY-DOX) and the skin was closed with surgical clips (Kent Scientific Corp). Mice were placed on a 37° C. micro-temp circulating pump and blanket during surgery and recovery period and given a pre-emptive dose of 0.2 mg/kg Meloxicam along with a dose of 0.1 mg/kg Buprenorphine immediately after surgery, and one additional dose of Meloxicam 24 h later. Wound clips were removed 10 days after surgery and mice were monitored twice a week.

Mice were then analyzed for graft function at various time points by performing in vivo [3]. After fasting the mice for 16 h overnight, a glucose challenge was performed by intraperitoneal injection (IP) 2 g D-(+)-glucose/1 kg body weight and blood was collected both pre-injection and 30 min post-injection of glucose through facial vein puncture. Serum was separated out using Microvettes (Sarstedt) to then measure human insulin levels using the Human Ultrasensitive ELISA kit. Kidney grafts were dissected from the mice, fixed in 4% paraformaldehyde overnight, embedded in paraffin, and sectioned for the histological analysis as described above.

All animal experiments were performed in accordance with Harvard University International Animal Care and Use Committee (IACUC) regulations.

### RNA Analysis

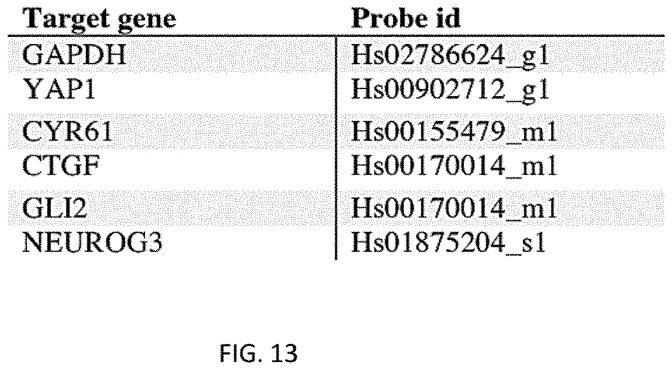

The total RNA was isolated using the Direct-ZOL Miniprep kit (Zymo Research), and RNA was stored at −80° C. in RNA storage solution (Invitrogen) until subsequent analysis. For real-time PCR, cDNA was synthesized using the SuperScript II Reverse Transcriptase kit (Invitrogen) and anchored oligo-dT primers (Invitrogen). TaqMan Fast Universal-based real-time PCR (Life Technologies) was performed in a ABI 7900HT PCR system (Applied Biosystems), with commercially available TaqMan gene expression assay probes (Thermo Scientific) with GAPDH as an internal normalization control (FIG. 13). For NanoString analysis, 50-100 ng of RNA was hybridized to a custom nCounter XT probe set and processed using the NanoString prep station and nCounter (NanoString Technologies). Gene expression levels were determined with NanoString nSolver software with default parameters and normalized with the expression of five housekeeping genes (ITCH, RPL15, RPL19, TCEB1, and UBE2D3). Nanostring data have been deposited into figshare under DOI accession code 10.6084/m9.figshare.7670531.

Western Blot Analysis

Dispersed cells were lysed in RIPA buffer (Thermo Scientific), and protein concentration was measured using the BCA Protein Assay kit (Thermo Scientific). In total, 5-10 μg of protein extracts were separated by AnyKD Mini-Protein precast gels (Bio-Rad) and transferred to nitrocellulose membranes (Bio-Rad). Membranes were blocked in 3% BSA+0.1% Tween 20 TBS for 30 min at RT and then incubated with the following primary antibodies overnight at 4° C.: rabbit anti-YAP (Cell Signaling; 14074S) and mouse anti-GAPDH (Millipore; MAB374) as the loading control. After washing, the membranes were incubated with HRP-conjugated secondary antibodies for 1 h at RT, and then incubated in chemiluminescent ECL detection reagent (VWR) for signal detection and development.

Statistical Analysis

Statistical analysis was performed using unpaired two-sided t tests, unless stated otherwise. For all the experiments included in this study, three or more biological replicates were performed using stage-matched controls as a reference. The Bonferroni-Dunn method was used for adjustment of multiple comparison analysis of qPCR and Nanostring data using GraphPad PRISM 7. Box plots, bar graphs, and heatmaps were generated with GraphPad PRISM 7 and R.

### REFERENCES

1. Alejandro, R. et al. Long-term function (6 years) of islet allografts in type 1 diabetes. Diabetes 46, 1983-1989 (1997).
2. Scharp, D. et al. Insulin independence after islet transplantation into type I diabetic patient. Diabetes 39, 515-518 (1990).
3. Pagliuca, F. et al. Generation of functional human pancreatic β cells in vitro. Cell 159, 428-439 (2014).
4. Rezania, A. et al. Reversal of diabetes with insulin-producing cells derived in vitro from human pluripotent stem cells. Nat. Biotechnol. 32, 1121-1133 (2014).
5. Jennings, R., Berry, A., Strutt, J., Gerrard, D. & Hanley, N. Human pancreas development. Dev 142, 3126-3137 (2015).
6. Pagliuca, F. & Melton, D. How to make a functional β-cell. Dev 140, 2472-2483 (2013).
7. Gradwohl, G., Dierich, A., LeMeur, M. & Guillemot, F. Neurogenin 3 is required for the development of the four endocrine lineages of the pancreas. PNAS 94, 1607-1611 (2000).
8. Gu, G., Dubauskaite, J. & Melton, D. A. Direct evidence for the pancreatic lineage: NGN3+ cells are islet progenitors and are distinct from duct progenitors. Development 129, 2447-2457 (2002).
9. McGrath, P. S., Watson, C. L., Ingram, C., Helmrath, M. A. & Wells, J. M. The basic helix-loop-helix transcription factor NEUROG3 is required for the development of the human endocrine pancreas. Diabetes 64, 2497-2505 (2015).
10. Zhou, Q. et al. A multipotent progenitor domain guides pancreatic organogenesis. Dev. Cell. 13, 103-114 (2007).
11. Azzarelli, R. et al. Multi-site neurogenin3 phosphorylation controls pancreatic endocrine differentiation. Dev. Cell. 41, 274-286 (2017).
12. Krentz, N. A. J. et al. Phosphorylation of NEUROG3 links endocrine differentiation to the cell cycle in pancreatic progenitors. Dev. Cell. 41, 129-142 (2017).
13. Miyatsuka, T., Kosaka, Y., Kim, H. & German, M. S. Neurogenin3 inhibits proliferation in endocrine progenitors by inducing Cdkn1a. PNAS 108, 185-190 (2011).
14. Sander, M. et al. Homeobox gene NKX6.1 lies downstream of NKX2.2 in the major pathway of beta-cell formation in the pancreas. Development 127, 5533-5540 (2000).
15. Stoffers, D. A., Ferrer, J., Clarke, W. L. & Habener, J. F. Early-onset type-II diabetes mellitus (MODY4) linked to IPF1. Nat. Genet. 17, 138-139 (1992).
16. Kroon, E. et al. Pancreatic endoderm derived from human embryonic stem cells generates glucose-responsive insulin-secreting cells in vivo. Nat. Biotechnol. 26, 443-452 (2008).
17. Nostro, M. C. et al. Stage-specific signaling through TGFb family members and WNT regulates patterning and pancreatic specification of human pluripotent stem cells. Development 138, 861-871 (2011).
18. Nostro, M. C. et al. Efficient generation of NKX6-1+ pancreatic progenitors from multiple human pluripotent stem cell lines. Stem Cell Rep. 4, 591-604 (2015).
19. Rezania, A. et al. Maturation of human embryonic stem cell-derived pancreatic progenitors into functional islets capable of treating pre-existing diabetes in mice. Diabetes 61, 2016-2029 (2012).
20. Hansen, C. G., Moroishi, T. & Guan, K. L. YAP and TAZ: a nexus for Hippo signaling and beyond. Trends Cell Biol. 25, 499-513 (2015).
21. Dong, J. et al. Elucidation of a universal size-control mechanism in Drosophila and mammals. Cell 130, 1120-1133 (2007).
22. Gao, T. et al. (2013). Hippo signaling regulates differentiation and maintenance in the exocrine pancreas. Gastroenterology 144, 1543-1553 (2013).
23. George, N. M., Day, C. E., Boerner, B. P., Johnson, R. L. & Sarvetnick, N. E. Hippo signaling regulates pancreas development through inactivation of Yap. Mol. Cell. Biol. 32, 5116-5128 (2012).
24. Cebola, I. et al. TEAD and YAP regulate the enhancer network of human embryonic pancreatic progenitors. Nat. Cell Biol. 17, 615-626 (2015).
25. Mamidi, A. et al. Mechanosignalling via integrins directs fate decisions of pancreatic progenitors. Nature; doi.org/10.1038/s41586-018-0762-2 (2018).
26. Liu-Chittenden, Y. et al. Genetic and pharmacological disruption of the TEAD-YAP complex suppresses the oncogenic activity of YAP. Genes Dev. 26, 1300-1305 (2012).
27. Rosenbluh, J. et al. β-Catenin-driven cancers require a YAP1 transcriptional complex for survival and tumorigenesis. Cell 151, 1457-1473 (2012).
28. George, N. M., Boerner, B. P., Mir, S. U. R., Guinn, Z. & Sarvetnick, N. E. Exploiting expression of hippo effector, Yap, for expansion of functional islet mass. Mol. Endocrinol. 29, 1594-1607 (2015).
29. Yuan, T. et al. Proproliferative and antiapoptotic action of exogenously introduced YAP in pancreatic β cells. JCI Insight 1, 1-14 (2016).
30. Morvaridi, S., Dhall, D., Greene, M. I., Pandol, S. J. & Wang, Q. Role of YAP and TAZ in pancreatic ductal adenocarcinoma and in stellate cells associated with cancer and chronic pancreatitis. Sci. Rep. 5, 16759 (2015).
31. Deng, Y. et al. Yap1 regulates multiple steps of chondrocyte differentiation during skeletal development and bone repair. Cell Rep. 14, 2224-2237 (2016).
32. Goto, H. et al. Loss of Mob1a/b in mice results in chondrodysplasia due to YAP1/TAZ-TEAD-dependent repression of SOX9. Development 145, dev159244-11 (2018).
33. Heallen, T. et al. Hippo pathway inhibits Wnt signaling to restrain cardiomyocyte proliferation and heart size. Science 332, 458-461 (2011).
34. Zhang, Z. W. et al. miR-375 inhibits proliferation of mouse pancreatic progenitor cells by targeting YAP1. Cell. Physiol. Biochem. 32, 1808-1817 (2013).
35. Musah, S. et al. Substratum-induced differentiation of human pluripotent stem cells reveals the coactivator YAP is a potent regulator of neuronal specification. PNAS 111, 13805-13810 (2014).
36. Salomoni, P. & Calegari, F. Cell cycle control of mammalian neural stem cells: putting a speed limit on G1. Trends Cell Biol. 20, 233-243 (2010).
37. Seymour, P. A. et al. SOX9 is required for maintenance of the pancreatic progenitor cell pool. PNAS 104, 1865-1870 (2007).
38. Shih, H. P. et al. A gene regulatory network cooperatively controlled by Pdx 1 and Sox9 governs lineage allocation of foregut progenitor cells. Cell Rep. 13, 1-12 (2015).
39. Song, S. et al. Hippo coactivator YAP1 upregulates SOX9 and endows esophageal cancer cells with stem-like properties. Cancer Res. 74, 4170-4182 (2014).
40. Chetty, S. et al. A simple tool to improve pluripotent stem cell differentiation. Nat. Methods 10, 553-556 (2013).

1. A method of producing a stem cell-derived beta cell comprising contacting at least one endocrine progenitor cell during a beta cell differentiation protocol with a YAP modulator, wherein the at least one endocrine progenitor cell differentiates into at least one stem cell-derived beta cell.

2-18. (canceled)

19. A population of stem cell-derived beta cells produced according to the method of claim 1.

20-23. (canceled)

24. A method comprising increasing or decreasing YAP expression during a beta cell differentiation protocol.

25-33. (canceled)

34. A composition comprising a PDX1-positive, NKX6.1-positive cell and a YAP inhibitor.

35. The composition of claim 34, wherein the cell is a PDX1-positive, NGN3-positive cell.

36. The composition of claim 34, wherein the YAP inhibitor is verteporfin.

37. The composition of claim 34, wherein the YAP inhibitor is a shRNA that reduces YAP expression.

38. The composition of claim 34, wherein the composition further comprises a TGF-beta signaling pathway inhibitor.

39. The composition of claim 38, wherein the TGF-beta signaling pathway inhibitor is Alk5 inhibitor II.

40. The composition of claim 34, wherein the composition further comprises a thyroid hormone signaling pathway activator.

41. The composition of claim 34, wherein the composition further comprises a SHH pathway inhibitor.

42. The composition of claim 41, wherein the SHH pathway inhibitor is Sant1.

43. The composition of claim 34, wherein the composition further comprises a RA signaling pathway activator.

44. The composition of claim 43, wherein the RA signaling pathway activator is retinoic acid.

45. The composition of claim 34, wherein the composition further comprises a gamma-secretase inhibitor.

46. The composition of claim 45, wherein the gamma-secretase inhibitor is XXI.

47. The composition of claim 34, wherein the composition further comprise at least one growth factor from the epidermal growth factor (EGF) family.

48. The composition of claim 47, wherein the at least one growth factor from the epidermal growth factor (EGF) family is Betacellullin.

49. The composition of claim 34, wherein the composition further comprises a TGF-beta signaling pathway inhibitor and a thyroid hormone signaling pathway activator.

50. The composition of claim 34, wherein the composition further comprises a TGF-beta signaling pathway inhibitor and a SHH pathway inhibitor.

51. The composition of claim 34, wherein the composition further comprises a gamma-secretase inhibitor.

52. The composition of claim 34, wherein the composition further comprises Alk5 inhibitor II and Sant1.

53. The composition of claim 34, wherein the composition further comprises Alk5 inhibitor II and a gamma-secretase inhibitor.

54. A composition comprising a PDX1-positive, NKX6.1-negative cell and a YAP activator.

55. The composition of claim 54, wherein the YAP activator is XMU-MP-1 or CEP-701/lestaurtanib.
